(19) United States
(12) Patent Application Publication
(10) Pub. No.: US 2026/0134231 A1
(43) Pub. Date: May 14, 2026

(54) LARGE LANGUAGE MODEL (LLM)-BASED QUESTION ANSWERING METHOD AND APPARATUS FOR TRADITIONAL CHINESE MEDICINE (TCM), DEVICE, AND MEDIUM

(71) Applicant: Innovation Center of Yangtze River Delta, ZJU, Jiaxing (CN)

(72) Inventors: Xiaohui FAN, Jiaxing (CN); Xin SHAO, Jiaxing (CN); Yizheng DAI, Jiaxing (CN); Jinlu ZHANG, Jiaxing (CN)

(73) Assignee: Innovation Center of Yangtze River Delta, ZJU, Jiaxing (CN)

(21) Appl. No.: 19/234,320

(22) Filed: Jun. 11, 2025

(30) Foreign Application Priority Data
Nov. 8, 2024 (CN) 202411585625.9

(51) Int. Cl. G06F 40/51; G06F 40/289

(52) U.S. Cl. CPC G06F 40/51; G06F 40/289

(57) ABSTRACT

A large language model (LLM)-based question answering method and apparatus for traditional Chinese medicine (TCM), a device, and a medium are provided. The LLM-based question answering method obtains multi-source TCM knowledge data from a book, literature, a network platform, and a TCM dataset; constructs diversified instruction data suitable for different scenarios, overcoming a shortcoming of affecting performance and reliability of a model due to possible non-professional or inaccurate information introduced through collection by using a chat generative pre-trained transformer (ChatGPT) application programming interface (API); performs training based on a Baichuan2-7B-Chat model to obtain a question answering model for TCM, realizing a process from pre-training (PT) to supervised fine-tuning (SFT); and sets different verification metrics based on different application scenarios to verify the model, overcoming a shortcoming of limiting evaluation accuracy of the model due to a single or subjective evaluation metric.

### CROSS REFERENCE TO THE RELATED APPLICATIONS

This application is based upon and claims priority to Chinese Patent Application No. 202411585625.9, filed on Nov. 8, 2024, the entire contents of which are incorporated herein by reference.

### TECHNICAL FIELD

The present disclosure relates to the field of medical question answering, and in particular, to a large language model (LLM)-based question answering method and apparatus for traditional Chinese medicine (TCM), a device, and a medium.

### BACKGROUND

Large language models (LLMs) are deep learning models trained based on a large amount of textual data, and are intended to understand and generate natural language text. These models are typically based on a transformer architecture and can capture language complexity and diversity. Their applications in the field of biomedicine are becoming increasingly widespread, especially in the field of TCM. These models have brought many innovations to research, application, and dissemination of TCM by understanding and generating the natural language text. At present, a most common application is to use a large model to analyze massive textual data of TCM, such as literature, ancient books, and modern research papers, and extract key information such as names, natures, tastes, meridian tropisms, efficacy, usage and dosage, and compatibility contraindications of TCM from the textual data to construct a TCM knowledge base. Recently, some researchers have developed a large model named CMLM-ZhongJing for question answering of knowledge of TCM and auxiliary diagnosis and treatment. This large model is based on tabular data of gynecological prescriptions of TCM, and generates instruction data for 15 scenarios by setting a specific prompt template. Finally, the instruction data is obtained through fine-tuning by the large model. However, training data is typically collected by relying on chat generative pre-trained transformer (ChatGPT) application programming interface (API) (chat application learning platform) to construct an aligned dataset. Relying on the ChatGPT API may introduce non-professional or inaccurate information, affecting performance and reliability of the model. In addition, existing large models for TCM adopt a single evaluation metric, and most of them are verified through subjective evaluation, which seriously limits evaluation accuracy of the large models for TCM.

### SUMMARY

An objective of the present disclosure is to provide a large language model (LLM)-based question answering method and apparatus for TCM, a device, and a medium, which can improve accuracy and reliability of question answering for TCM.

To achieve the above objective, the present disclosure provides the following technical solutions.

According to a first aspect, the present disclosure provides an LLM-based question answering method for TCM, including:
constructing a TCM knowledge database, where the TCM knowledge database includes multi-source TCM knowledge data obtained from a book, literature, a network platform, and a TCM dataset;
generating unsupervised data and instruction data based on the TCM knowledge database, where application scenarios of the instruction data include a TCM knowledge base, a choice question, reading comprehension, entity extraction, medical case diagnosis, and TCM or prescription recommendation;
constructing a Baichuan2-7B-Chat model, where the Baichuan2-7B-Chat model is obtained by improving a transformer decoder, where an improvement method includes: replacing a LayerNorm layer in the transformer decoder with an RMSNorm layer, replacing an absolute position embedding layer in the transformer decoder with a rotary position embedding layer, and replacing a ReLu activation function in the transformer decoder with a SwiGLU activation function;
performing unsupervised pre-training (PT) on the Baichuan2-7B-Chat model by using the unsupervised data to obtain a pre-trained Baichuan2-7B-Chat model;
performing supervised training on the pre-trained Baichuan2-7B-Chat model by using the unsupervised data and the instruction data to obtain a trained Baichuan2-7B-Chat model;
setting a verification metric for each of the application scenarios, and verifying the trained Baichuan2-7B-Chat model by using the verification metric; and
performing TCM question answering by using a verified Baichuan2-7B-Chat model.

According to a second aspect, the present disclosure provides an LLM-based question apparatus for TCM, including a backend and a frontend, where
the frontend is configured to interact with a client; and
the backend is configured to perform question answering by using the above LLM-based question answering method for TCM.

According to a third aspect, a computer device is provided, including a memory, a processor, and a computer program stored in the memory and executable on the processor, where the processor executes the computer program to implement the above LLM-based question answering method for TCM.

According to a fourth aspect, a computer-readable storage medium is provided, storing a computer program thereon, where the computer program is executed by a processor to implement the above LLM-based question answering method for TCM.

According to a fifth aspect, a computer program product is provided, including a computer program, where the computer program is executed by a processor to implement the above LLM-based question answering method for TCM.

According to specific embodiments provided in the present disclosure, the present disclosure achieves the following technical effects:

The present disclosure provides an LLM-based question answering method and apparatus for TCM, a device, and a medium. The present disclosure obtains multi-source TCM knowledge data from a book, literature, a network platform, and a TCM dataset; constructs diversified instruction data suitable for different scenarios, directly overcoming a shortcoming of affecting performance and reliability of a model due to possible non-professional or inaccurate information introduced through collection by using a ChatGPT API (large model); performs training based on a Baichuan2-7B-Chat model (base model of parameters of Bachman-7B) to obtain a question answering model for TCM, realizing a process from PT to supervised fine-tuning (SFT); and sets different verification metrics based on different application scenarios to verify the model, overcoming a shortcoming of limiting evaluation accuracy of the model due to single or subjective evaluation metrics. The present disclosure improves accuracy and reliability of TCM question answering.

### BRIEF DESCRIPTION OF THE DRAWINGS

To describe the technical solutions in the embodiments of the present disclosure or in the prior art more clearly, the following briefly describes the accompanying drawings required for the embodiments. Apparently, the accompanying drawings in the following description show merely some embodiments of the present disclosure, and a person of ordinary skill in the art may still derive other accompanying drawings from these accompanying drawings without creative efforts.

FIG. 1 is a flowchart of an LLM-based question answering method for TCM according to an embodiment of the present disclosure;

FIG. 2 is a schematic diagram of a training process of an LLM according to an embodiment of the present disclosure;

FIG. 3 shows a comparison result of Example 1 according to an embodiment of the present disclosure;

FIG. 4 shows a comparison result of Example 2 according to an embodiment of the present disclosure;

FIG. 5 shows a comparison result of Example 3 according to an embodiment of the present disclosure;

FIG. 6 shows a comparison result of Example 4 according to an embodiment of the present disclosure.

FIG. 7 shows a comparison result of Example 5 according to an embodiment of the present disclosure;

FIG. 8 shows a comparison result of Example 6 according to an embodiment of the present disclosure;

FIG. 9 shows a comparison result of Example 7 according to an embodiment of the present disclosure;

FIG. 10 shows a comparison result of Example 8 according to an embodiment of the present disclosure; and

FIG. 11 is a schematic structural diagram of a computer device according to an embodiment of the present disclosure.

### REFERENCE NUMERALS

A1 is indicated as follows:

### Case 1: Case Report-Based Diagnosis

A2 is indicated as follows:

Before the age of 30, the patient had experienced coughing, expectoration, and wheezing without apparent triggers, and had consulted a doctor at a local hospital for symptomatic treatment. The aforementioned symptoms had been improved after the symptomatic treatment. Thereafter, the patient had undergone recurrent episodes of coughing, expectoration, and wheezing, which were often worsened due to catching cold or a seasonal transition. These symptoms persisted for more than three months each year. The patient had sought medical care at local hospitals multiple times due to exacerbations and had been diagnosed with “chronic obstructive pulmonary disease (COPD)”. After symptomatic treatments such as medications for resolving phlegm, relieving wheezing, and diminishing inflammation were applied, the condition had been improved. 4 days ago, the patient experienced aggravated chest tightness and wheezing after catching cold, which were exacerbated after physical activities. The patient also had a cough with a moderate amount of yellow phlegm and a low-grade fever at night, with a maximum body temperature of 37.8° C. and no significant chest pain. The patient was self-administered with oral drugs, but the specific details of the drugs are unknown. In order to seek further diagnosis and treatment, the patient visited our hospital for medical care and was admitted by the outpatient department to the inpatient department under the diagnosis of “acute exacerbation of COPD”. During the course of disease, the patient did not experience nausea, vomiting, hemoptysis, chest pain, and edema of the lower limbs, and had a fair appetite, an average sleep quality, and normal bowel movements and urination. The patient was conscious, and had a passable mental state, a moderate body, clear speeches, rosy lips, and normal skin without any rashes or spots. The skull had a normal size and shape, with no sunken eye sockets, no yellow staining of the sclera, normal helixes, and no auricular fistulas or sores. The neck was symmetrical, and did not have bulging veins or goiter and scrofula. The chest was symmetrical, and had normal apical pulse. The abdomen was flat, and did not have any masses or lumps. The nails were rosy. There was no edema in the lower limbs. The tongue was pale red and had a white coating. There was a taut and slippery pulse.

### Pattern Differentiation and Treatment Methods?

A3 is indicated as follows:

### [Syndrome] Damp-Phlegm Obstructing the Lungs

[Etiology] This refers to a category of symptoms such as dull and cloudy coughing, profuse phlegm, white and dense tongue coating, and a soft and slippery pulse, which arise due to the phenomena that there is the spleen's failure in transportation and transformation, and food and water cannot be converted into essential nutrients transported upwards to nourish the lungs, but accumulate as phlegm-turbidity in the lungs, causing lung qi-stagnation and upward rebellion and obstruction.

### [Diagnosis] Asthma

[Treatment principle]: Eliminating dampness and phlegm, and regulating qi and relieving cough. [Recommended] Common Chinese medicinal herbs, including Pinellia ternata, Buddha's hand, processed Rhizoma Arisaematis, etc.; and prescription drugs: Dingchuan Zhike Syrup, Er Chen Wan, Wuhuang Yangyin granules, etc.

A4 is indicated as follows:

According to the symptoms and medical history of the patient, the patient is preliminarily diagnosed with an acute exacerbation of COPD. The treatment plan includes the following aspects:

1. Infection control: The infection in the patient needs to be controlled timely, which can be achieved through an antibiotic therapy.
2. Phlegm reduction and cough relief: The patient should take drugs for reducing phlegm and relieving cough, such as Ambroxol or Ambroxol hydrochloride.
3. Anti-inflammatory therapy: The patient should be administered with anti-inflammatory drugs, such as cephalosporins and penicillins.
4. Oxygen therapy: The patient requires an oxygen therapy, such as oxygen inhalation or supplemental oxygen.
5. Supportive therapy: The patient should focus on rest, have adequate sleep, and avoid overexertion.

Based on the condition of the patient, the following Chinese patent medicines or prescriptions are recommended.

1. Feining granules: This drug has the effects of clearing heat, detoxifying, reducing phlegm, and relieving cough, and is suitable for patients with acute exacerbations of COPD.
2. Feining Tablets: This drug has the effects of clearing heat, detoxifying, reducing phlegm, and relieving cough, and is suitable for patients with acute exacerbations of COPD.
3. Feining Tablets: This drug has the effects of clearing heat, detoxifying, reducing phlegm, and relieving cough, and is suitable for patients with acute exacerbations of COPD.
4. Feining Tablets: This drug has the effects of clearing heat, detoxifying, reducing phlegm, and relieving cough, and is suitable for patients with acute exacerbations of COPD.
5. Feining Tablets: This drug has the effects of clearing heat, detoxifying, reducing phlegm, and relieving cough, and is suitable for patients with acute exacerbations of COPD. \n\n It should be noted that a TCM treatment requires individualized formulation based on the specific situation of a patient. It is recommended that the patient takes TCM under the guidance of a qualified physician.

A5 is indicated as follows:

Based on the medical history and symptoms of the patient, it can be preliminarily determined that the patient suffers from an acute exacerbation of COPD. According to the syndrome differentiation system of TCM, it can be preliminarily summarized as phlegm and blood stasis obstructing the lung, qi stagnation, and heat accumulation, indicating the syndromes of internal dryness due to phlegm-turbidity and heat transformation from qi stagnation. Manifestations are aggravated coughing, expectoration, and wheezing, yellow phlegm, fever, chest tightness, and shortness of breath.

In terms of treatment, it is recommended to adopt the principles of resolving phlegm, relieving asthma, clearing heat, and detoxifying to alleviate the internal stagnation of phlegm-turbidity, the airway obstruction, and the inflammation. The following TCM prescriptions are recommended:

###TCM prescriptions:

1. Rhizoma Anemarrhenae (30 g), Semen Sinapis albae (10 g), Radix Peucedani (10 g), and Radix Glycyrrhizae (5 g): clearing heat and eliminating phlegm, and moistening lungs and relieving asthma.
2. Semen armeniacae amarum (15 g), Cortex Mori (10 g), and Flos Farfarae (10 g): moistening lungs and reducing phlegm, and relieving cough and asthma.
3. Radix Polygalae (10 g), Pedicellus Melo (10 g), and Rhizoma Bletillae (10 g): drying dampness and dispelling phlegm, promoting lung dispersion and relieving chest oppression, and clearing heat and detoxifying.
4. Radix asparagi (15 g), Spica prunellae (10 g), and Fructus Forsythiae (10 g): clearing heat and detoxifying, and clearing heat from blood and promoting lung dispersion.

###Precautions:

1. During the medication period, the uncooked and cold foods and the overwork should be avoided, and the emotional stability should be maintained.
2. If the phlegm is thick and difficult to expectorate, an appropriate amount of Loquat Extract Syrup or Fritillaria Loquat Syrup may be taken as an adjunct.
3. It is recommended to closely monitor changes in the condition. If the symptoms such as severe difficult breathing and persistent high fever occur, it is necessary to seek medical advice promptly.

The above TCM prescriptions are for reference only, and a professional TCM doctor needs to be consulted for the specific dosage and course of treatment. Wish the patient a speedy and full recovery!

A6 is indicated as follows:

Before the age of 30, the patient had experienced coughing, expectoration, and wheezing without apparent triggers and had consulted a doctor at a local hospital for symptomatic treatment. The aforementioned symptoms had been improved after the symptomatic treatment. Thereafter, the patient had undergone recurrent episodes of coughing, expectoration, and wheezing, which were often worsened due to catching cold or a seasonal transition. These symptoms persisted for more than three months each year. The patient had sought medical care at local hospitals multiple times due to exacerbations and had been diagnosed with “COPD”. After symptomatic treatments such as medications for resolving phlegm, relieving wheezing, and diminishing inflammation were applied, the condition had been improved. 4 days ago, the patient experienced aggravated chest tightness and wheezing after catching cold, which were exacerbated after physical activities. The patient also had a cough with a moderate amount of yellow phlegm and a low-grade fever at night, with a maximum body temperature of 37.8° C. and no significant chest pain. The patient was self-administered with oral drugs, but the specific details of the drugs are unknown. In order to seek further diagnosis and treatment, the patient visited our hospital for medical care and was admitted by the outpatient department to the inpatient department under the diagnosis of “acute exacerbation of COPD”.

A7 is indicated as follows:

### **Pattern Differentiation:**

Cough, expectoration, and wheezing, which are worsened after catching cold and persist for many years: Lung qi deficiency and weakened defensive qi
Cough with a moderate amount of yellow phlegm: Internal obstruction due to phlegm-heat
Low-grade fever at night: Stagnation due to heat pathogen
Pale-red tongue with a white coating: Qi deficiency and weakened defensive qi Taut and slippery pulse: Internal obstruction due to phlegm-heat
Syndrome pattern: Lung qi deficiency, weakened defensive qi, and internal obstruction due to phlegm-heat
Treatment principle: Replenishing qi and securing the exterior, and resolving phlegm and relieving asthma.

### Recommended Prescriptions:

Xiao Qinglong Jia Shigao Tang
Ephedra: 9 g; Cinnamon Twig: 9 g; gypsum: 30 g; Semen Armeniacae Amarum: 9 g; Radix Glycyrrhizae: 6 g; Asarum: 3 g; Zingiber officinale: 9 g; and Chinese date: 3 dates.

### Modifications:

If the coughing is aggravated violently, Belamcanda Rhizome and Flos Farfarae are added.
If there is excessive yellow phlegm, Pinellia ternata and Poria are added.
If the wheezing is significant, Perilla seeds and Fritillaria are added.
If the fever is significant, Radix Scutellariae and Rhizoma coptidis are added.

A8 is indicated as follows:

Based on the medical history and symptoms provided by the patient, the patient is preliminarily diagnosed with an acute exacerbation of COPD. A pulmonary function test is proposed for the patient to assess the pulmonary function and the severity of illness. Moreover, it is necessary to rule out complications such as pulmonary infection and bronchitis. In terms of treatment, the targeted medication should be adopted based on the specific condition of the patient, such as antibiotics, bronchodilators, and glucocorticoids. It is also recommended that the patient adopts certain lifestyle changes, such as quitting smoking, avoiding pollutants, and taking exercise. In addition, it is necessary to enhance nutrition, get enough sleep, and maintain a prominent state of mind. It should be noted that the above recommendations are for reference only, and the specific treatment plan should be adjusted according to the specific circumstances of the patient.

A9 is indicated as follows:

Symptomatic treatments such as medications for resolving phlegm, relieving wheezing, and diminishing inflammation

A10 is indicated as follows:

During the course of disease, the patient did not experience nausea, vomiting, hemoptysis, and chest pain, and

had an average sleep quality, normal bowel movements and urination, a passable mental state, a moderate body, normal skin, no sunken eye sockets and yellow staining of the sclera, normal helixes without auricular fistulas or sores, a symmetrical neck without goiter or scrofula, a symmetrical chest, a pale-red tongue, a taut and slippery pulse, no edema in the lower limbs, and a pale-red tongue.

B1 is indicated as follows:

### Case 2: Reading Comprehension

B2 is indicated as follows:

<Context> Ms. Wang, a reader from Jiangsu, asks: “I am 40 years old and have great skin, and the skin on the face is not dry. But the corners of my mouth keep cracking, and it hurts a lot when I open my mouth. What's going on and what should I do?” Wang Junlan, Deputy Chief Physician of TCM in the Gynecology Department at Nanjing Integrated Traditional Chinese and Western Medicine Hospital, replies: “Many cases of cracked and painful corners of the mouth are not significantly related to the overall skin state or whether the skin on the face is dry. From the perspective of TCM, this condition is related to the spleen. In the TCM, it is said that the spleen opens into the mouth and its vitality is reflected in the lips. Thus, invigorating the spleen is the key to preventing dry and chapped lips. In winter, there is a dry climate, which makes the lips prone to dryness and cracking. The primary measure in preventing and treating dry and chapped lips is to break the lip licking habit. It is also important to drink plenty of water and consume lots of fresh vegetables, pears, water chestnuts, and other foods that have fluid-generating and yin-nourishing effects. The foods that are cold in nature and can easily damage the spleen qi should be avoided, such as bitter melons and cucumbers. The foods that are rich, greasy, heavy, or cloying and can easily hinder the spleen's transformative and transport functions should be avoided, such as milk and soft-shelled turtles. The foods that promote qi movement and digestion and break down food stagnation and can easily deplete the spleen qi should be avoided, such as radishes and hawthorns. In the TCM, it is believed that the spleen is the foundation of postnatal existence and the source of qi and blood production. When the eating and drinking are in moderation and the strong spleen and stomach are maintained, the transformative and transport functions are normal, and the blood production is continuous and abundant. Therefore, in daily life, it is worth trying a combination of food and herbal remedies under the guidance of a physician of TCM. For example, Chinese dates and Angelica sinensis can be appropriately taken to nourish the blood and strengthen the spleen. Astragalus root and Pseudostellaria root can be used in combination to boost qi and fortify the spleen. Poria and Atractylodes macrocephala can be further administered to invigorate the spleen and promote the blood production. However, it is important to note that most of these drugs are rich and cloying, and should not be taken when there are symptoms such as fever caused by exogenous pathogens and loose or watery stools. In winter, taking an herbal paste for nourishing the spleen under the guidance of a TCM practitioner after proper syndrome differentiation can also be an excellent choice.”<question> What is the cause of cracked and painful corners of the mouth?

B3 is indicated as follows:

Many cases of cracked and painful corners of the mouth are not significantly related to the overall skin state or whether the skin on the face is dry. From the perspective of TCM, this condition is related to the spleen. In the TCM, it is said that the spleen opens into the mouth and its vitality is reflected in the lips. Thus, invigorating the spleen is the key to preventing dry and chapped lips. In winter, there is a dry climate, which makes the lips prone to dryness and cracking.

B4 is indicated as follows:

The cracked and painful corners of the mouth may be related to the spleen function in the body. According to the perspective of TCM, the spleen opens into the mouth. Thus, invigorating the spleen can prevent dry and chapped lips. In winter, there is a dry climate, which makes the lips prone to dryness and cracking. The following prevention and treatment measures are recommended: breaking the lip licking habit; drinking plenty of water; consuming foods that have fluid-generating and yin-nourishing effects, such as fresh vegetables, pears, and water chestnuts; and avoiding foods that are cold, rich, or cloying or can easily harm the spleen qi. A combination of food and herbal remedies can also be tried. For example, medicinal herbs such as Chinese dates, Angelica sinensis, Astragalus root, and Pseudostellaria root can be taken to nourish and strengthen the spleen qi. However, it is important to note that most of these drugs are rich and cloying, and should not be taken when there are symptoms such as fever caused by exogenous pathogens and loose or watery stools. According to the specific syndrome differentiation of a TCM practitioner, an herbal paste can also be considered to nourish the spleen.

B5 is indicated as follows:

This issue is complex because different types of pain correspond to varying causes and treatments. Generally, various types of pain may be related to the liver, the lungs and spleen, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys, the lungs and kidneys . . . .

B6 is indicated as follows:

There may be many causes for cracked and painful corners of the mouth, such as angular cheilitis, cheilitis, angular dermatitis, or angular rash. These conditions are often related to factors such as skin health, oral hygiene, malnutrition, and stress. In your case, the symptoms may be caused by spleen deficiency. In the TCM, it is said that the spleen opens into the mouth and its vitality is reflected in the lips. Thus, invigorating the spleen is the key to preventing dry and chapped lips. In winter, there is a dry climate, which makes the lips prone to dryness and cracking. The primary measure in preventing and treating dry and chapped lips is to break the lip licking habit. It is also important to drink plenty of water and consume lots of fresh vegetables, pears, water chestnuts, and other foods that have fluid-generating and yin-nourishing effects. The foods that are cold in nature and can easily damage the spleen qi should be avoided, such as bitter melons and cucumbers. The foods that are rich, greasy, heavy, or cloying and can easily hinder the spleen's transformative and transport functions should be avoided, such as milk and soft-shelled turtles. The foods that promote qi movement and digestion and break down food stagnation and can easily deplete the spleen qi should be avoided, such as radishes and hawthorns. In the TCM, it is believed that the spleen is the foundation of postnatal existence and the source of qi and blood production. When the eating and drinking are in moderation and the strong spleen and stomach are maintained, the transformative and transport functions are normal, and the blood production is continuous and abundant. Therefore, in daily life, it is worth trying a combination of food and herbal remedies under the guidance of a physician of TCM. For example, Chinese dates and Angelica sinensis can be appropriately taken to nourish the blood and strengthen the spleen. Astragalus root and Pseudostellaria root can be used in combination to boost qi and fortify the spleen. Poria and Atractylodes macrocephala can be further administered to invigorate the spleen and promote the blood production. However, it is important to note that most of these drugs are rich and cloying, and should not be taken when there are symptoms such as fever caused by exogenous pathogens and loose or watery stools. In winter, taking an herbal paste for nourishing the spleen under the guidance of a TCM practitioner after proper syndrome differentiation can also be an excellent choice. If you have other symptoms or concerns, it is recommended to seek a medical attention promptly.

B7 is indicated as follows:

This condition is related to the spleen. In the TCM, it is said that the spleen opens into the mouth and its vitality is reflected in the lips.

B8 is indicated as follows:

There can be many causes of cracked and painful corners of the mouth, including poor oral hygiene, malnutrition, vitamin

B2 deficiency, vitamin B6 deficiency, vitamin B12 deficiency, oral inflammation, dry mouth, etc. It is recommended for you to see a doctor in the department of stomatology for examination to determine the specific cause. In addition, the cracked and painful corners of the mouth can be alleviated by keeping the oral cavity clean, drinking plenty of water, eating lots of fresh vegetables and fruits, and avoiding bad habits like lip licking.

B9 is indicated as follows:

There are various reasons for chapped corners of the mouth on the face. It is generally believed that the chapped corners of the mouth are resulted from the lack of lubrication at the corners of the mouth. In the oral cavity, there is a certain temperature difference locally and a relatively-low humidity. In addition, with the increase of an age, the skin gradually becomes rough, and the stratum corneum will be formed, which makes the secretion of sebaceous glands weakened to cause cracked corners of the mouth. The cracked corners of the mouth can be triggered by many other factors, primarily including mental stress, depression, anxiety, overexertion, and poor dietary habits, which all may exacerbate the symptom.

B10 is indicated as follows:

What is the cause of cracked and painful corners of the mouth?

C1 is indicated as follows:

### Case 3: Recommendation of Chinese Patent Medicines or Prescriptions

C2 is indicated as follows:

Chinese patent medicines with a similar chemical composition to Mingmu Shangqing Wan?

C3 is indicated as follows:

The Chinese patent medicines with a similar chemical composition to Mingmu Shangqing Wan include Qingxin Mingmu Shangqing wan, Niuhuang Shangqing Capsule, Niuhuang Shangqing Pill, Niuhuang Shangqing Tablet, and Mingmu Jili Pill.

C4 is indicated as follows:

Chinese patent medicines with a similar chemical composition to Mingmu Shangqing Wan include:

1. Mingmu Shangqing Wan: The main ingredients in Mingmu Shangqing Wan include Astragalus root, Rhizoma coptidis, Phellodendron amurense, Gardenia jasminoides, Fructus Forsythiae, Radix isatidis, Chrysanthemum, mulberry leaves, Chrysanthemum, peppermint, Radix Glycyrrhizae, etc.
2. Mingmu Shangqing Granules: The main ingredients in Mingmu Shangqing Granules include Astragalus root, Rhizoma coptidis, Phellodendron amurense, Gardenia jasminoides, Fructus Forsythiae, Radix isatidis, Chrysanthemum, mulberry leaves, Chrysanthemum, peppermint, Radix Glycyrrhizae, etc.
3. Mingmu Shangqing Capsules: The main ingredients in Mingmu Shangqing Capsules include Astragalus root, Rhizoma coptidis, Phellodendron amurense, Gardenia jasminoides, Fructus Forsythiae, Radix isatidis, Chrysanthemum, mulberry leaves, Chrysanthemum, peppermint, Radix Glycyrrhizae, etc.

It should be noted that the above prescriptions have different pharmaceutical ingredients and action mechanisms, and it is necessary to consult a doctor or a pharmacist of TCM for advice before specific use.

C5 is indicated as follows:

What are Chinese patent medicines with a similar chemical composition to Mingmu Shangqing Wan?What kind of Chinese patent medicines are these?

### ###Question:

What are the characteristics of these Chinese patent medicines?

C6 is indicated as follows:

### None

C7 is indicated as follows:

Chinese patent medicines with a similar chemical composition to Mingmu Shangqing Wan include Chuan Xiong Cha Tiao San, Butian Dazao Wan, Simiao Wan, etc.

1. Chuan Xiong Cha Tiao San mainly contains Ligusticum chuanxiong, Angelica sinensis, Rehmannia glutinosa, Paeonia lactiflora, Aconitum carmichaelii, Angelica dahurica, Radix aucklandiae, and Radix saposhnikoviae. Chuan Xiong Cha Tiao San can be used for headache, dizziness, flushed face and red ears, distending pain in both hypochondriac regions, and even chest tightness, vomiting, restlessness, a red tongue with a thin coating, and a wiry and rapid pulse that are caused by hyperactivity of liver Yang. For treating headaches caused by a cold, the following herbs can be added: Cinnamon Twig, Platycodon grandiflorus, Fructus Arctii, raw Radix Glycyrrhizae, Fructus Forsythiae, Semen Armeniacae Amarum, Radix Peucedani, Zingiber officinale, and Radix Glycyrrhizae.
2. Butian Dazao Wan mainly contains Ginseng, Ephedra, Poria, Radix salviae miltiorrhizae, Ligusticum chuanxiong, and Pheretima aspergillum. Butian Dazao Wan is mainly used to treat dizziness, blurred vision, emaciation with sallow complexion, abdominal pain, limb weakness, palpitations, shortness of breath, women's metrorrhagia and metrostaxis, urinary dribbling, etc. that are caused by spleen deficiency, liver stagnation, and blood stasis.
3. Simiao Wan mainly contains Radix bupleuri, Notopterygium incisum, Angelica pubescens, Fructus viticis, Angelica dahurica, Asarum, Ephedra, Rhizoma atractylodis, Semen Sinapis Albae, and Radix Glycyrrhizae. Simiao Wan can be used for lassitude of the loin and legs, numbness of the limbs, heavy hands and feet, spasm of the limbs, unfavorable flexion and extension, and difficulty in walking that are caused by rheumatism.

C8 is indicated as follows:

Based on similar chemical compositions to Mingmu Shangqing Wan, some Chinese patent medicines with a similar composition to Mingmu Shangqing Wan can be recommended, such as Qingrejiedu oral liquid and Qingnao Yizhi wan. However, before recommending any medicines, it is necessary to understand the condition, physical status, and other medications of a patient to ensure that the recommended medicines are appropriate for the patient. Can you tell me more about your situation? Finally, it is advised that the patient must follow the doctor's advice and the instructions of a drug when choosing the drug, and should also pay attention to the side effects and contraindications of the drug when in use.

C9 is indicated as follows:

Mingmu Shangqing Wan is a TCM prescription for treating ophthalmic diseases. Mingmu Shangqing Wan has the effect of clearing heat and improving eyesight and the effect of nourishing yin and blood. Similar Chinese patent medicines are recommended based on a similar chemical composition to Mingmu Shangqing Wan. The following are some possible Chinese patent medicines with a similar chemical composition to Mingmu Shangqing Wan:

1. **Shenggan Mingmu Wan**: Shenggan Mingmu Wan is a Chinese patent medicine for treating ophthalmic diseases. Shenggan Mingmu Wan has the effects of improving the eyesight and protecting the liver, and is similar to Mingmu Shangqing Wan to some extent.
2. **Ming Mu Di Huang Wan**: Ming Mu Di Huang Wan is a Chinese patent medicine for nourishing yin and brightening the eyes. Ming Mu Di Huang Wan contains medicinal materials such as Radix rehmanniae, and can be used for reference and comparison.
3. **Ming Mu Yin**: Ming Mu Yin is a Chinese patent medicine for clearing heat, brightening eyes, nourishing yin, and moistening dryness. Ming Mu Yin may also have similar components to Mingmu Shangqing Wan to some degree.

The above Chinese patent medicines are just some examples that may have a similar chemical composition to Mingmu Shangqing Wan. When these Chinese patent medicines are specifically selected, the personalized formulation and selection are required according to the condition and body constitution of the patient. It is recommended that the patient should consult a doctor or a pharmacist of TCM before taking a medicine to acquire more professional advices.

C10 is indicated as follows:

Chinese patent medicines with a similar chemical composition to Mingmu Shangqing Wan include **Huyan Anshen Wan**, **Yigan Qingmu Wan**, **Bugan Mingmu Wan**, etc. These Chinese patent medicines may share some pharmacological actions and indications with Mingmu Shangqing Wan. However, it is necessary to consult a TCM expert for more accurate information on the specific efficacy and functions of these Chinese patent medicines.

### DETAILED DESCRIPTION OF THE EMBODIMENTS

The technical solutions in the embodiments of the present disclosure are clearly and completely described below with reference to the accompanying drawings in the embodiments of the present disclosure. Apparently, the described embodiments are only some rather than all of the embodiments of the present disclosure. All other embodiments obtained by a person of ordinary skill in the art based on the embodiments of the present disclosure without creative efforts shall fall within the protection scope of the present disclosure.

To make the above objectives, features, and advantages of the present disclosure more obvious and easy to understand, the present disclosure will be further described in detail with reference to the accompanying drawings and specific implementations.

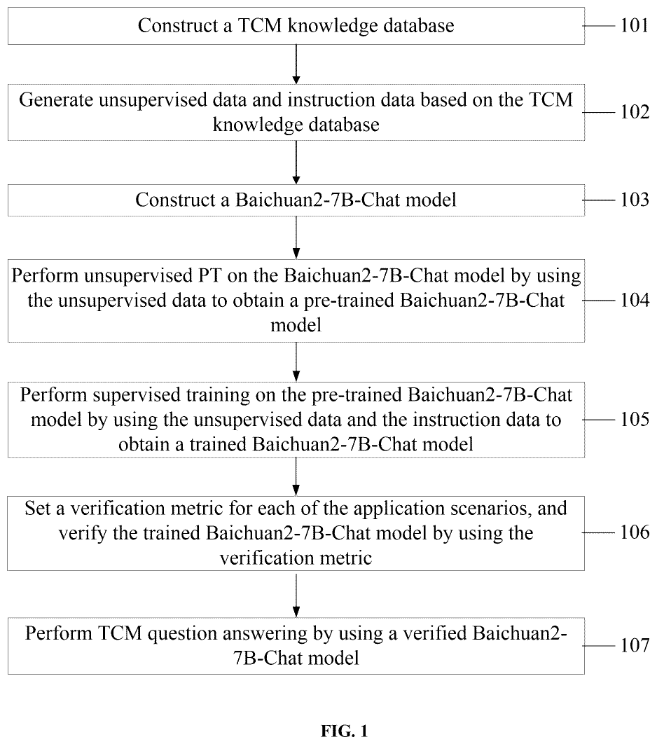

In an exemplary embodiment, as shown in FIG. 1, an LLM-based question answering method for TCM is provided. The LLM-based question answering method is executed by a computer device, and specifically, may be executed separately by the computer device such as a terminal or a server, or may be executed jointly by the terminal and the server. In this embodiment of the present disclosure, an example in which the LLM-based question answering method is applied to the server is used for description, and the following steps 101 to 107 are included:

Step 101: Construct a TCM knowledge database, where the TCM knowledge database includes multi-source TCM knowledge data obtained from a book, literature, a network platform, and a TCM dataset.

Step 102: Generate unsupervised data and instruction data based on the TCM knowledge database, where application scenarios of the instruction data include a TCM knowledge base, a choice question, reading comprehension, entity extraction, medical case diagnosis, and TCM or prescription recommendation.

Step 103: Construct a Baichuan2-7B-Chat model, where the Baichuan2-7B-Chat model is obtained by improving a transformer decoder, where an improvement method includes: replacing a LayerNorm layer in the transformer decoder with an RMSNorm layer, replacing an absolute position embedding layer in the transformer decoder with a rotary position embedding layer, and replacing a ReLu activation function in the transformer decoder with a SwiGLU activation function.

Step 104: Perform unsupervised PT on the Baichuan2-7B-Chat model by using the unsupervised data to obtain a pre-trained Baichuan2-7B-Chat model.

Step 105: Perform supervised training on the pre-trained Baichuan2-7B-Chat model by using the unsupervised data and the instruction data to obtain a trained Baichuan2-7B-Chat model.

Step 106: Set a verification metric for each of the application scenarios, and verify the trained Baichuan2-7B-Chat model by using the verification metric.

Step 107: Perform TCM question answering by using a verified Baichuan2-7B-Chat model.

According to the above steps 101 to 107, the multi-source TCM knowledge data is obtained from the book, the literature, the network platform, and the TCM dataset; the diversified instruction data suitable for different scenarios is constructed, directly overcoming a shortcoming of affecting performance and reliability of a model due to possible non-professional or inaccurate information introduced through collection by using a ChatGPT API (large model); the training is performed based on the Baichuan2-7B-Chat model (base model of parameters of Baichuan-7B) to obtain a question answering model for TCM, realizing a process from PT to SFT; and different verification metrics are set based on the different application scenarios to verify the model, overcoming a shortcoming of limiting evaluation accuracy of the model due to single or subjective evaluation metrics. The present disclosure improves accuracy and reliability of the TCM question answering.

Construction of a large model for TCM relies on fine-tuning of the instruction data to a great extent. This process is mainly implemented by training and optimizing the model on a large number of datasets related to TCM, which can enhance understanding and application capabilities of the model in the field of TCM. However, although the fine-tuning is an important step of training an LLM, model PT, high-quality instruction data construction, and multidimensional model evaluation also play an indispensable role. Therefore, this embodiment of the present disclosure utilizes diversified instruction datasets to train the base model, including PT and fine-tuning stages, and conducts multidimensional evaluation to construct a TCMChat system. The TCMChat system is mainly applied to answer basic knowledge of TCM, perform intelligent recommendation and medical case diagnosis, and the like. Therefore, structured and unstructured data is collected from different sources. The data is classified into the unsupervised data and the supervised instruction data based on a certain strategy, and the training is performed on a cluster server. The trained model is compared with other models to quantify accuracy of the model under different test sets.

Construction of the instruction data is an essential step before fine-tuning the model. Recently, there have been numerous literature reports on methods for constructing an instruction set of TCM based on raw data. For example, BenTso constructs an instruction dataset based on a knowledge atlas and then performs the fine-tuning on Chinese-LLaMA. CMLM-ZhongJing uses professional tabular data and strictly sets a specific prompt template to generate instruction data for 15 scenarios. A fine-tuned model has an ability to infer prescription data and diagnostic thinking logic of TCM. TCMChat adopts a more comprehensive and diversified strategy in its collection. In addition to TCM books and open-source data, the TCMChat also introduces multi-source data such as literature knowledge, web platform data, and databases, and trains the multi-source data by using a full-parameter approach. In order to more scientifically evaluate performance of the TCMChat, this embodiment of the present disclosure establishes a multidimensional, objective, and comprehensive evaluation system, which includes but is not limited to basic metrics such as model accuracy, a recall, and an F1 score, and also introduces mean reciprocal rank (MRR), BERTSocre, normalized discounted cumulative gain (nDCG), and other advanced metrics. Actual value of the model can be more comprehensively reflected by comprehensively evaluating these metrics.

In another exemplary embodiment, data used to construct the TCM knowledge database in the step 101 is from the book, the literature, the network platform, and the TCM dataset. The constructed TCM knowledge database in the step 101 covers a wide range of dimensions, including the book, the literature, web crawler information, professional literature, and an open-source dataset, aiming to comprehensively and deeply explore rich knowledge in the field of TCM. Specifically, the following aspects are covered:

Books and materials: TCM books are an important part of the field of TCM. They not only carry theoretical knowledge of TCM, but also record rich clinical practice experience and drug information. Resources including national standards, medical textbooks, and medical cases provide a solid foundation for theoretical, practical, and historical research on TCM.

Disease and syndrome information: Information sources of the Chinese medical information query platform are extensive and authoritative, including TCM information released by authoritative institutions such as the State Administration of Traditional Chinese Medicine and the China Academy of Traditional Chinese Medicine. Information on the platform is strictly reviewed and authenticated and its accuracy and reliability are ensured. Through the Chinese medical information query platform (TCM-DaYi), detailed disease and syndrome records can be obtained, thereby greatly enriching a clinical application databased of the TCM.

Herb and prescription data: As a comprehensive resource database for TCM, the Encyclopedia of Traditional Chinese Medicine (ETCM) website is aimed at integrating pharmacological information of TCM and providing correlation data between a component of TCM and a target to help researchers understand a pharmacological mechanism of TCM. Through the ETCM website, detailed herb and prescription data can be collected, providing valuable resources for research on the formulaology and the herbology of TCM.

Academic literature abstract: As a largest comprehensive academic resource database in China, the China National Knowledge Infrastructure (CNKI) integrates various types of academic resources such as journals, dissertations, conference papers, newspapers, yearbooks, patents, standards, and scientific and technological achievements, providing convenient academic resource retrieval and download services for scholars, researchers, educators, and students. Through the CNKI, literature abstracts of TCM and related fields can be downloaded, providing important support for deepening and expanding a theory of TCM.

BaiduBaiKe: BaiduBaiKe contains rich TCM information, covering various aspects such as basic concepts, types, origins, collection, processing, medicinal theories, compatibility, contraindications, dosage and usage, and naming of TCM. BaiduBaike text data (BaiduBaike) is obtained from a GitHub platform, and includes popularized knowledge of TCM.

Reading comprehension and named entity recognition of TCM: The Alibaba Cloud Tianchi Platform is a scientific research data platform open to the public by Alibaba Group. It is jointly provided by Alibaba Group's business team and external research institutions, covering more than ten industries such as e-commerce, entertainment, logistics, healthcare, transportation, industry, natural science, and energy, as well as classic artificial intelligence fields such as data mining, machine learning, computer vision, natural language processing, and decision intelligence. A Traditional Chinese Medicine-Reading Comprehension (TCM-RC) dataset and a Traditional Chinese Medicine-Named Entity Recognition (TCM-NER) dataset are downloaded from the Alibaba Cloud Tianchi Platform, which are crucial for improving performance of a natural language processing model in the field of TCM.

TCM syndrome: A Traditional Chinese Medicine-Syndrome Differentiation (TCM-SD) dataset is the first publicly available TCM-SD dataset collected based on actual scenarios, which contains a large number of clinical records from the real world, and covers various TCM syndromes. Through the GitHub (TCM-SD) platform, a syndrome TCM dataset is compiled, providing strong support for syndrome diagnosis and classification research.

Herb and prescription recommendation: ShenNong_TCM_Dataset is a large-scale dataset dedicated to the field of TCM. It is based on an open-source knowledge atlas of TCM and calls ChatGPT and other models to generate instruction data about TCM. In order to enhance a capability of a TCM recommendation system, the ShenNong_TCM_Dataset is introduced, which provides a rich data foundation for personalized herb or prescription recommendation.

In another exemplary embodiment, in the step 102, the unsupervised data mainly comes from the book, the BaiduBaiKe, the TCM-DaYi, literature on the CNKI, and the ShenNong_TCM_Dataset. Firstly, an optical character recognition method is used to extract text information from a PDF format of the book, and the extracted text is manually proofread and edited, including correcting a spelling error, and adding or removing a punctuation mark and a paragraph format. When a literature abstract is processed, text mining should be applied to remove an HTML tag that may be included in content and correct a symbol error to ensure data accuracy and readability. The TCM-DaYi and the ShenNong_TCM_Dataset are processed by using a simple segmentation method. A method for constructing the supervised instruction data is a systematic process, which can ensure that the TCMChat can perform a specific task. Specifically, the following different construction strategies are used:

1) Creating a human-machine interaction instruction; 2) converting a template into a text format; and 3) collecting an open-source dataset. Data obtained based on these strategies are manually verified and screened to generate six basic scenarios: the TCM knowledge base, the choice question, the reading comprehension, the entity extraction, the medical case diagnosis, and the TCM or prescription recommendation.

In another exemplary embodiment, the Baichuan2-7B-Chat model in the step 103 adopts a decoder architecture of a transformer and has made the following improvements based on previous work: 1) replacing the LayerNorm layer with the RMSNorm layer; 2) replacing the absolute position embedding with the rotary position embedding; and 3) replacing the ReLu activation function with the SwiGLU.

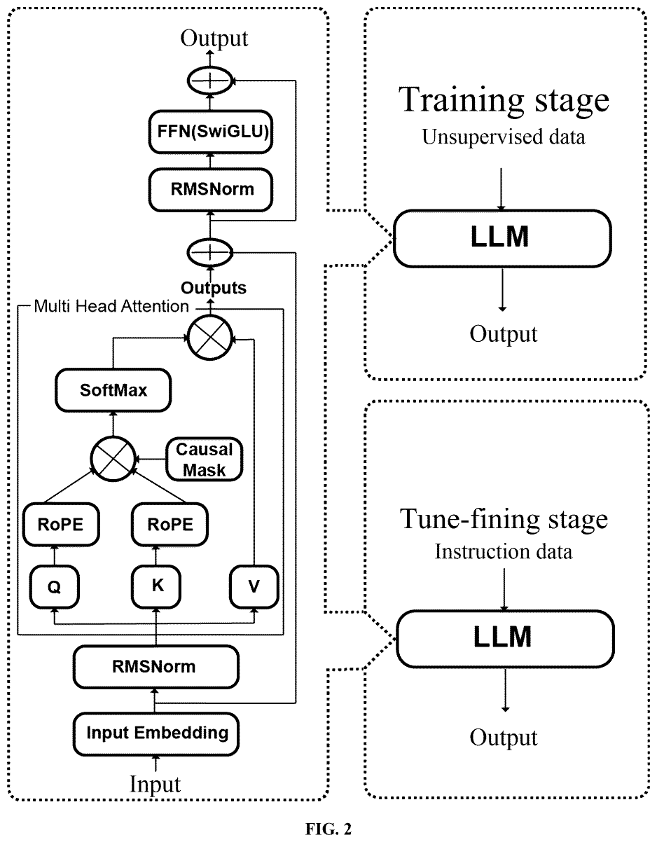

In another exemplary embodiment, as shown in FIG. 2, a left part of FIG. 2 is a network structure of the LLM, namely the Baichuan2-7B-Chat model, which includes an input embedding layer for converting a text string into a text vector. A multi-head attention layer is used to enhance a capability of the model to capture more levels of information. The RMSNorm layer is used to solve problems such as a large or small parameter and gradient explosion caused by the large or small parameter. A feedforward layer (feedforward neural network (FFN) (SwiGLU)) is used to perform nonlinear transformation and mapping on a vector representation of each position. In FIG. 2, Outputs represents an output of the multi-head attention layer, SoftMax represents the activation function, RoPE represents the rotary position embedding, and Q, K, and V respectively Query, Key, Value in an attention mechanism. A right part of FIG. 2 is a process of model training, including the PT and the fine-tuning. Both the PT and the fine-tuning use a same network structure. The PT mainly uses the unsupervised data, and after the PT, the instruction data is used for the fine-tuning.

Specifically, the steps 104 and 105 are specifically as follows:

In the PT stage (corresponding to the step 104), a causal language model (CLM) is used. For a given input sequence, the model only uses prior information of the sequence to predict a probability of a next word or character. This is expressed in a mathematical representation of a loss function as follows:

$${L}_{CLM}^{( A )} = - \frac{1}{\vert A \vert} {\sum }_{k = 1}^{\vert A \vert} log {P}_{θ} ( {A}_{k} \vert {A}_{< k} ) \qquad ( 1 )$$

In the above formula,

$${L}_{CLM}^{( A )}$$

LIM represents the loss function, A represents a sentence sequence in the unsupervised data, |A| represents a length of the sentence sequence, and P_{θ}(Aₖ|A_{<k}) represents an operation of predicting a probability of a k^{th} word Aₖ by using a word A_{<k} before a k^{th} word when a parameter of the Baichuan2-7B-Chat model is θ.

In the SFT stage, a generative language model uses the same loss function used in the PT stage to perform the fine-tuning. A significant difference lies in concatenating question and answer text into a long sequence and adding a special separator to mark question and answer boundaries as an input. Therefore, an overfitting risk of the model for a specific task is reduced by using the instruction data to enhance adaptability and generalization capabilities of a deep learning model, lowering a learning rate, and increasing a batch size.

In the steps 104 and 105, considering that the LLM typically requires a large amount of graphics processing unit (GPU) memory to store various parameters during training (such as an intermediate activation and a weight), using only one GPU cannot meet a training requirement. Therefore, an entire experimental process is conducted in parallel by using 8*NVIDA-A800.In addition, in order to increase the batch size during the model training and prevent GPU out of memory (OOM), this embodiment of the present disclosure uses a DeepSpeed technology to reduce a usage ratio of the GPU memory. At present, DeepSpeed parallelism methods include ZeRO-1, ZeRO-2, ZeRO-3, ZeRO-Offload, and ZeRO-Infinity. The ZeRO-1 divides optimizer parameters into a plurality GPUs, and a process on each GPU is only responsible for updating a parameter of the Gubser on the ZeRO-1, the ZeRO-2 also segments gradient information. Only a part of the gradient information is needed to update a model weight, and model parameters is still saved on each computing device. A calculated gradient of a layer is aggregated through collective communication. Aggregated gradient information is saved only by a device that needs the aggregated gradient information, and is released by other devices that do not need the aggregated gradient information. Based on the ZeRO-1 and the ZeRO-2, the ZeRO-3 also segments the model weight and assigns segmented weights to different devices. The segmented weights are synchronized through the collective communication only when needed, and are released immediately after calculation. The ZeRO-Offload distributes the model parameters on a central processing unit (CPU) and the GPU, and calculates some gradients through the CPU to reduce memory usage. But this also incurs a certain computational overhead. The ZeRO-Infinity is an extension of the ZeRO-3. The ZeRO-Infinity allows use of an NVMe solid-state drive to expand the GPU memory and CPU memory to train the large model. Therefore, the DeepSpeed technology can efficiently train models with parameters ranging from 1.5B to several hundred B, and provide significant acceleration for these models.

For example, the TCMChat has different parameters for the PT and SFT stages. In the PT stage, the learning rate is 2e-4, the batch size is 32 for each GPU, and a maximum context length is 1024 tokens. In the SFT stage, full fine-tuning is used. The learning rate is adjusted to 2e-5, the batch size is 16 for each GPU, and the maximum context length is limited to 1024 tokens. An optimizer of the model uses AdamW and specifies a learning rate of 1e-4. A training experiment uses 8 NVIDIA A100 GPUs and uses the ZeRO-2 and ZeRO-3 methods of the DeepSpeed technology to enhance the training, thereby minimizing the memory usage and speeding up the process. To ensure training stability, problems of gradient explosion and learning rate decay are alleviated by reducing a loss by half.

In another exemplary embodiment, to ensure scientific and accurate research, instruction data under five scenarios are randomly extracted. In addition, in order to compare quality of the model training, different closed-source and open-source models are selected as a baseline, and different parameter sizes can externally reflect generative capacities of the models.GPT-3.5-Turbo is an optimized version of GPT-3.5 (175 billion parameters), but its exact number of parameters has not been disclosed yet. Gemini-Pro is a large-scale language model with 100 billion yet. Gemini-med, Bentsao-literature, HuatuoGPT, and the CMLM-zhongjing all have 7 billion parameters, and the same is true for the TCMChat. A smallest model BenQue2 also has 6 billion parameters. Finally, the TCMChat is benchmarked against other large-scale language modeling algorithms to evaluate its performance in response to different scenarios. Specifically, in order to objectively compare performance of the TCMChat in the TCM question answering, five different types of evaluation datasets are constructed, involving the choice question, the reading comprehension, the entity extraction, the medical case diagnosis, and the TCM or prescription recommendation. In the different application scenarios, the verification metric in the step 106 is specifically as follows:

Choice question: The choice question covers knowledge of TCM and the prescription, and 500 pieces of data are separately extracted from knowledge of TCM and the prescription as a test set. For each question answering response in the test dataset, accuracy of a selected answer is calculated according to a following formula:

$$Accuracy = \frac{TP + TN}{TP + TN + FP + FN} \qquad ( 2 )$$

In the above formula, Accuracy represents the accuracy of the selected answer, and TP, TN, FP, and FN respectively represent a number of true positive cases, a number of false positive cases, a number of false negative cases, and a number of true negative cases.

Reading comprehension: 500 pieces of data are randomly selected as a test set for the reading comprehension, and their similarity is evaluated by using a bilingual evaluation understudy (BLEU) metric, a metric for evaluation of translation with explicit ordering (METEOR), a recall-oriented understudy for gisting evaluation (ROUGE) metric, and the BertScore metric.

The BLEU metric is used to quantify a similarity between a generated sentence and a reference sentence, and to evaluate sentence-level fluency by evaluating an n-gram overlapping degree. A specific formula is as follows:

$$BLEU = BP \times exp ( {\sum }_{n = 1}^{N} {W}_{n} \times log {P}_{n} ) \qquad ( 3 )$$

In the above formula, BLEU represents the BLEU metric; BP represents a penalty factor; N represents a size of a window for truncating continuous words; Wₙ represents a weight of an n^{th} phrase set within the window for truncating the continuous words; and Pₙ represents accuracy of the n^{th} phrase set, and the accuracy of the n^{th} phrase set is obtained by comparing various phrases in the n^{th} phrase set obtained by truncating generated text and reference text.

The METEOR is used as a software metric to evaluate text quality and complexity. Main formulas are as follows:

$$METEOR = ( 1 - Pen ) \times F \qquad ( 4 )$$

$$F = \frac{P \times R}{α \times P + ( 1 - α ) \times R} \qquad ( 5 )$$

$$Pen = γ * {( \frac{m}{ch} )}^{{β}_{1}} \qquad ( 6 )$$

In the above formulas, METEOR represents the METEOR, Pen represents a fragment penalty factor, F represents a harmonic mean of the accuracy and the recall, P represents accuracy of the generated text, R represents a recall of the generated text, a represents a parameter for adjusting weights of the P and the R, m represents a number of phrases allowed to be matched in the generated text, ch represents a number of phrases in the reference text, γ represents a proportional parameter, and f₁ represents a power parameter.

Then, the ROUGE metric measures an n-gram overlapping degree between a generated output and a reference abstract. In theory, response quality of the model is evaluated based on word matching in a longest common subsequence. The ROUGE metric mainly includes ROUGE-1, ROUGE-2, and ROUGE-L. Main formulas are as follows:

$$Rouge - N = \frac{{\sum }_{S ∈ x} {\sum }_{{gram}_{n} ∈ S} {Count}_{match ( {gram}_{n} )}}{{\sum }_{S ∈ x} {\sum }_{{gram}_{n}} Count ( {gram}_{n} )} \qquad ( 7 )$$

where Rouge−N represents a metric for performing abstract evaluation by taking the size N of the window for truncating the continuous words as a length, S represents the reference text in a reference text set, x represents the reference text set, gramₙ represents the n^{th} phrase set within the window for truncating the continuous words, Count_{match(gram}_{ₙ}₎ represents a number of successfully matched phrases in the gramₙ, and Count_{(gram}_{ₙ}₎ represents a total number of phrases contained in the gramₙ; and

$$ROUGE - L = \frac{( 1 + {β}_{2}^{2} ) \frac{LCS ( x , \hat{x} ) LCS ( x , \hat{x} )}{\begin{matrix}len ( x ) & len ( \hat{x} )\end{matrix}}}{\frac{LCS ( x , \hat{x} )}{len ( x )} + {β}_{2}^{2} \frac{LCS ( x , \hat{x} )}{len ( \hat{x} )}} \qquad ( 8 )$$

where ROUGE−L represents a metric for performing the abstract evaluation based on a length of a longest common subsequence of the reference text and the generated text, \hat{x} represents a generated text set, LCS(x,\hat{x}) represents a length of a longest common subsequence of the x and the \hat{x}, len( ) represents a length calculation formula, and β₂ represents an adjustment parameter.

The BertScore metric is calculated based on a cosine similarity between word vectors output by a BERT model, and consists of two core metrics: the precision and the recall. In the following description, the recall refers to a recall of an embedding of the generated text set. The precision measures a degree at which a word in the generated text matches a word in the reference text, while the recall measures a degree at which the word in the reference text matches the word in the generated text. Main formulas are as follows:

$$Precision = \frac{1}{\vert \hat{x} ′ \vert} {\sum }_{{\hat{x}}_{j} ∈ \hat{x} ′} {max}_{{x}_{i} ∈ x ′} ( {x}_{i}^{T} \hat{{x}_{J}} ) \qquad ( 9 )$$

$$Recall = \frac{1}{\vert x ′ \vert} {\sum }_{{x}_{i} ∈ x ′} {max}_{{\hat{x}}_{j} ∈ \hat{x} ′} ( {x}_{i}^{T} \hat{{x}_{J}} ) \qquad ( 10 )$$

$$BertScore ( F 1 - score ) = 2 * \frac{Precision * Recall}{Precision + Recall} \qquad ( 11 )$$

In the above formulas, Precision represents the precision, x′ represents an embedding of the reference text set, \hat{x}′ represents the embedding of the generated text set, \hat{x}ⱼ represents an embedding of a j^{th} sentence in the embedding of the generated text set, xᵢ represents an embedding of an i^{th} sentence in the embedding of the reference text set, the superscript T represents transposition, Recall represents the recall of the embedding of the generated text set, and BertScore(F1−score) represents the BertScore metric.

Entity extraction: For this scenario, 480 pieces of data are randomly selected as a test set, and evaluated by using the precision, the recall, and the BertScore metric. For a specific formula, reference may be made to the formulas (9), (10), and (11).

Medical case diagnosis: For this scenario, 500 pieces of data are randomly selected as a test set, and evaluated by using the accuracy (which refers to the accuracy of the selected answer in the subsequent description), the precision, the recall, and the BertScore metric. For a specific formula, reference may be made to the formulas (1), (9), (10), and (11).

TCM or prescription recommendation: 500 pieces of data are randomly selected as a test set, and evaluated by using the MRR, precision@K, and the nDCG. The MRR is a metric of performance of an information retrieval system. The MRR is used to evaluate ranking quality of relevant documents in a system result, and its formula is as follows:

$$MRR = \frac{1}{\vert Q \vert} {\sum }_{q = 1}^{\vert Q \vert} \frac{1}{{rank}_{q}} \qquad ( 12 )$$

In the above formula, MRR represents the MRR, |Q| represents a number of suggestions, and rank_{q} represents a q^{th} suggestion.

The precision@K refers to precision at which the system provides correct or relevant information in the first K recommendations or search results, and its formula is as follows:

$$Precision @ K = \frac{TP @ K}{TP @ K + FN @ K} \qquad ( 13 )$$

In the above formula, Precision@K represents the precision@K, TP@K represents a number of true positive cases in the first K recommendations, and FN@K represents a number of true negative cases in the first K recommendations.

The nDCG is a metric used to rank quality of a search result, and is used to measure the quality of the search result or quality of a recommendation list. It considers a correlation of each project and a position (ranking) of the project in the search result, and lowers the position because a user is more likely to focus on the top-ranked results. A formula is as follows:

$$nDCG = \frac{{\sum }_{{p}_{1} = 1}^{P ′} \frac{{2}^{{rel}_{{p}_{1}}} - 1}{{log}_{2} ( {p}_{1} + 1 )}}{{\sum }_{{p}_{2} = 1}^{\vert REL \vert} \frac{{2}^{{rel}_{{p}_{2}}} - 1}{{log}_{2} ( {p}_{2} + 1 )}} \qquad ( 14 )$$

In the above formula, nDCG represents the nDCG, P′ represents a recommendation list of the generated text, relₚ_{₁} represents a score of a correlation between a p₁^{th} recommendation in the P′ and a question, |REL| represents a recommendation list of the reference text, and relₚ_{₂} represents a score of a correlation between a p₂^{th} recommendation in the |REL| and the question.

Based on the same inventive concept, the embodiments of the present disclosure further provide an LLM-based question answering apparatus for TCM to implement the above LLM-based question answering method for TCM. A problem-solving implementation solution provided by the apparatus is similar to the implementation solution described in the above method. Therefore, for following specific limitations on one or more embodiments of the LLM-based question answering apparatus for TCM, reference may be made to the above limitations on the LLM-based question answering method for TCM, and details are not described herein again.

In order to visually demonstrate an inference effect of the TCMChat, the model is designed and deployed in the apparatus embodiment. Deploying the large model typically involves a backend (such as Flask) and a frontend (such as Vue). The Flask is mainly configured to load a model weight and develop an external API, while the Vue is mainly configured to create a user interaction page.

In an exemplary embodiment, an LLM-based question answering apparatus for TCM is provided, including a backend and a frontend. The frontend is configured to interact with a client, and the backend is configured to perform question answering by using the LLM-based question answering method for TCM in the above embodiments.

When TCMChat is deployed, the apparatus embodiment not only builds an efficient backend service through Flask to fast load and run a model weight, but also develops an easily extensible API to ensure that the frontend can smoothly request and process data. In addition, a Vue.js framework is used to build a frontend page, which provides rich component libraries and responsive data bindings, making a user interaction interface beautiful and smooth, and can intuitively display a real-time inference result and an interactive feedback of the TCMChat.

In another exemplary embodiment, in order to verify effectiveness of the above method and apparatus embodiments, the following examples are also provided.

### Example 1

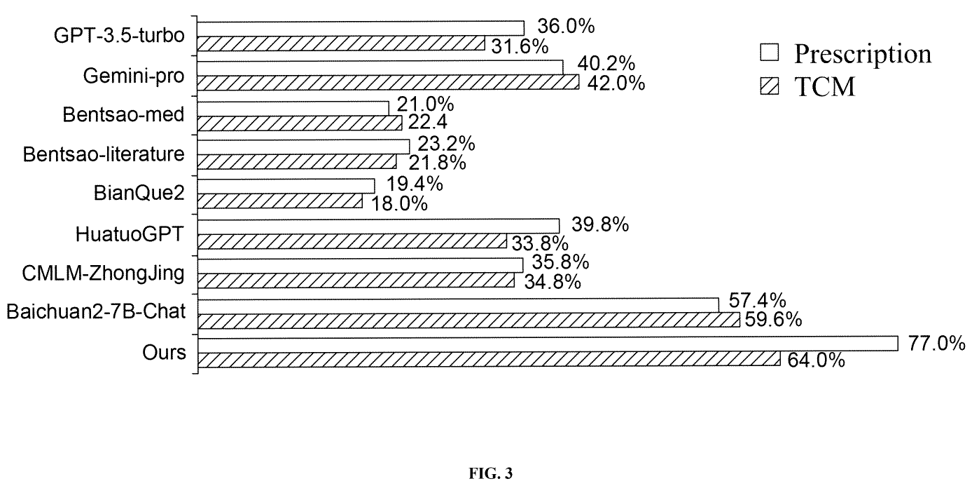

In this example, in order to evaluate performance of important models on the choice question, 500 pieces of TCM data and 500 pieces of prescription data are selected as a test set and input into each model in a few-shot mode. A model inference process does not rely on an output text response, but directly uses a maximum probability value of A, B, C, D, and E metrics in a last embedding layer of the model as a predicted answer. Finally, a key metric, namely the accuracy, is used to measure performance of each model. As shown in FIG. 3, the GPT-3.5-turbo, the Gemini-pro, the Bentsao-med, the Bentsao-literature, the BianQue2, the HuatuoGPT, the CMLM-zhongjing, and the Baichuan2-7B-chat are compared with the TCMChat (namely, Ours in FIG. 3) obtained by performing the supervised and unsupervised training on the Baichuan2-7B-Chat model in the present disclosure. Results show that the TCMChat performs better than other models and its accuracy is respectively 64.0% and 77.0% for TCM and the prescription, while accuracy of other models is all lower than 60% for TCM and the prescription.

### Example 2

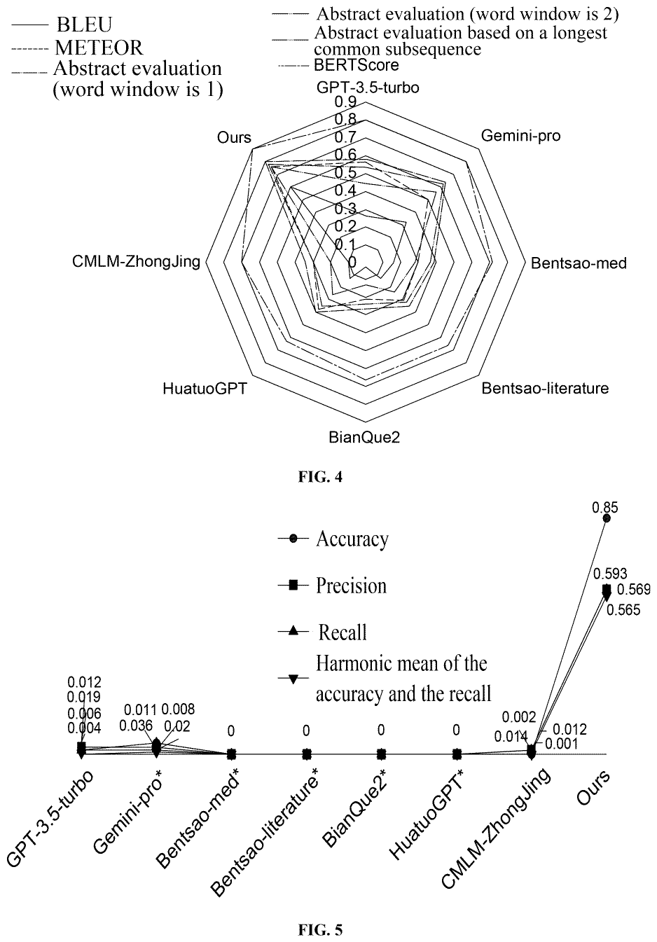

In this example, performance of the TCMChat (namely, Ours in FIG. 4) obtained by performing the supervised and unsupervised training on the Baichuan2-7B-Chat model in the present disclosure and performance of other large models (the GPT-3.5-turbo, the Gemini-pro, the Bentsao-med, the Bentsao-literature, the BianQue2, the HuatuoGPT, the CMLM-zhongjing, and the Baichuan2-7B-chat in FIG. 4) are evaluated on a dataset of 500 reading comprehension tests. This task requires the model to extract or infer relevant information from text and provide an answer to a question proposed in given text (usually referred to as a “context” or an “article”), and scores of the BLEU, METEOR, ROUGE, and BertScore metrics are obtained. Higher scores of the BLEU, ROUGE, METEOR, and BertScore metrics lead to better performance. As shown in FIG. 4, gray closed lines in FIG. 4 represent metric scores, which are 0, 0.1, 0.2, 0.3, 0.4, 0.5, 0.6, 0.7, 0.8, and 0.9 from inside to outside. Results show that the TCMChat in the present disclosure outperforms other models in terms of the BLEU (0.602), METEOR (0.749), ROUGE-1 (0.789), ROUGE-2 (0.753), ROUGE-L (0.785), and BertScore (0.894) metrics. It is worth noting that the Bentsao-med, the score of the BLEU metric is lower than 0.2 for the Bentsao-literature, the BianQue2, the HuatuoGPT, and the CMLM-zhongjing, which indicates that text generated by these models has low fluency. In summary, quality of the text generated by these models is not as good as that of text generated by the TCMChat in the present disclosure.

### Example 3

The medical case diagnosis has significance and value on many aspects in the field of TCM, and judgment of an evidence type can evaluate processing capabilities of various models in a medical case diagnosis task. As shown in FIG. 5, in a dataset of 500 medical case diagnosis tests, performance (accuracy: 0.850; precision: 0.593; recall (for the embedding of the generated text set): 0.565; F1-score (the harmonic mean of the accuracy and the recall): 0.569) of the TCMChat (namely, Ours in FIG. 5) in the present disclosure far exceeds that of other models. In addition, evaluation results of the Bentsao-med, the Bentsao-literature, the BianQue2, and the HuatuoGPT are all 0, which indicates that these models do not consider diagnosed cases or medical cases in medical practice when being constructed or trained. Although the GPT-3.5-turbo, the Gemini-pro, and the CMLM-zhongjing can determine an evidence type of a medical record, their performance values are relatively small and almost unable to perform medical record diagnosis. Therefore, the TCMChat algorithmically has more advantages in the medical record diagnosis.

### Example 4

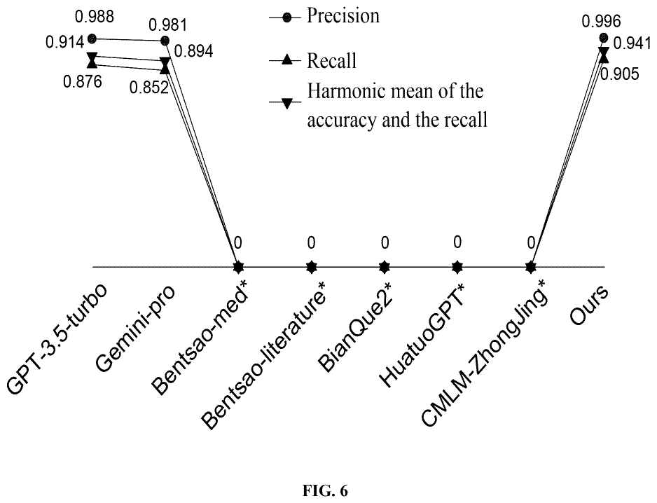

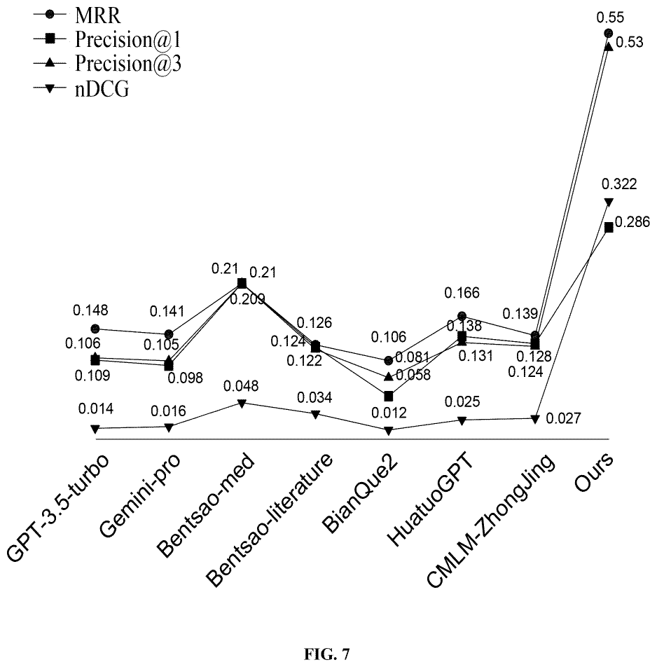

In this example, the entity extraction is evaluated. 480 pieces of data are randomly extracted from an instruction TCM dataset as a test set containing 13 entity types. Results show that, as shown in FIG. 6, the Bentsao-med, the Bentsao-literature, the BianQue2, the HuatuoGPT, and the CMLM-zhongjing are unable to extract an entity; the TCMChat (namely, Ours in FIG. 6) in the present disclosure performs well in the entity extraction (precision: 0.996; recall (for the embedding of the generated text set): 0.905; F1-score (the harmonic mean of the precision and the recall): 0.941); the GPT-3.5-turbo performs well in the entity extraction (precision: 0.988; recall: 0.876; and F1-score: 0.914); and the Gemini-pro performs well in the entity extraction (precision: 0.981; recall: 0.852; and F1-score: 0.894). It can be seen that the performance of the TCMChat in the present disclosure is slightly better than that of the GPT-3.5-turbo and the Gemini-pro.

### Example 5

Given importance of intelligent TCM recommendation, 500 pieces of data are randomly selected as a test set in this example, mainly involving corresponding TCM and prescriptions based on diseases, symptoms, and efficacy. In addition, due to an inability to evaluate consistency of output results of all models, in this example, output response text is parsed, and keywords of TCM and prescriptions are extracted from the text and sorted in a text output order. Evaluation results are shown in FIG. 7. Performance (MRR: 0.55; precision@1:0.286; precision@3:0.530; and nDCG: 0.322) of the TCMChat (namely, Ours in FIG. 7) in the present disclosure is significantly better than that of other large models (the GPT-3.5-turbo, the Gemini-pro, the Bentsao-med, the Bentsao-literature, the BianQue2, the HuatuoGPT, the CMLM-zhongjing, and the Baichuan2-7B-chat). Although the performance of the TCMChat can only reach a shallow level, after the PT and the SFT, the model has certain feasibility in the intelligent TCM recommendation.

In another exemplary embodiment, in order to more intuitively illustrate the performance of the TCMChat, cases in different scenarios are comparatively analyzed. Corresponding data mainly covers three scenarios: the medical case diagnosis, the reading comprehension, and the TCM or prescription recommendation, and is from a testing instruction set. A data input for the medical case diagnosis mainly includes patient information, medical treatment information, symptom description, and the like, while an input for the reading comprehension mainly includes contextual information and responsive questions. A data input for the TCM or prescription recommendation is attributes related to TCM or the prescription. All model outputs are in a text format and the following examples are provided.

### Example 6

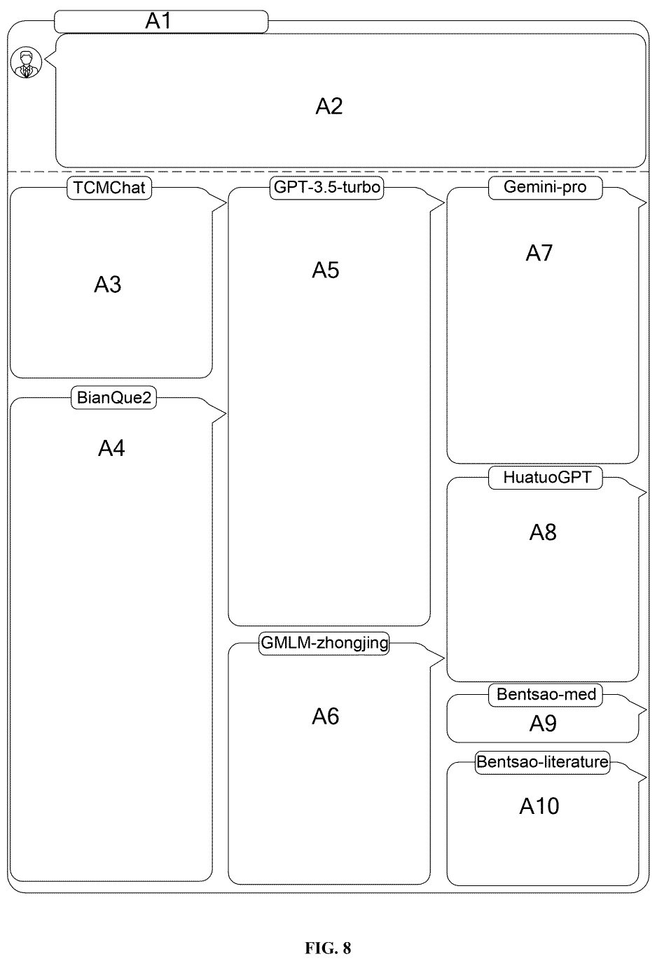

This example involves a case in which a medical record of TCM is diagnosed as “asthma”, as shown in FIG. 8. From FIG. 8, it can be seen that the Bentsao-literature, the Bentsao-med, and the CMLM-zhongjing make a response directly, without recognizing relevance of information of the medical record to TCM diagnosis and treatment. Responses from these models do not include relevant content or keywords to meet a user need, while the Bianque2 responds with some treatment suggestions, but repeatedly generates same content. The HuotuoGPT does not provide substantial diagnostic recommendations of TCM. The Gemini-pro responds with very detailed prescription compatibility, but cannot accurately determine a type of the medical record. Compared with other models, the GPT-3.5-turbo correctly determines the type of the medical record, as it regards that a treatment is “resolving phlegm, relieving asthma, clearing heat and detoxifying”. But a best treatment is not “clearing heat and detoxifying”, but “relieving a cough”. The TCMChat responds with the following information: (1) a syndrome type; (2) a diagnosis; (3) a treatment; and (4) a recommendation, which exhibits superior response logic and comprehension compared with the baseline models.

### Example 7

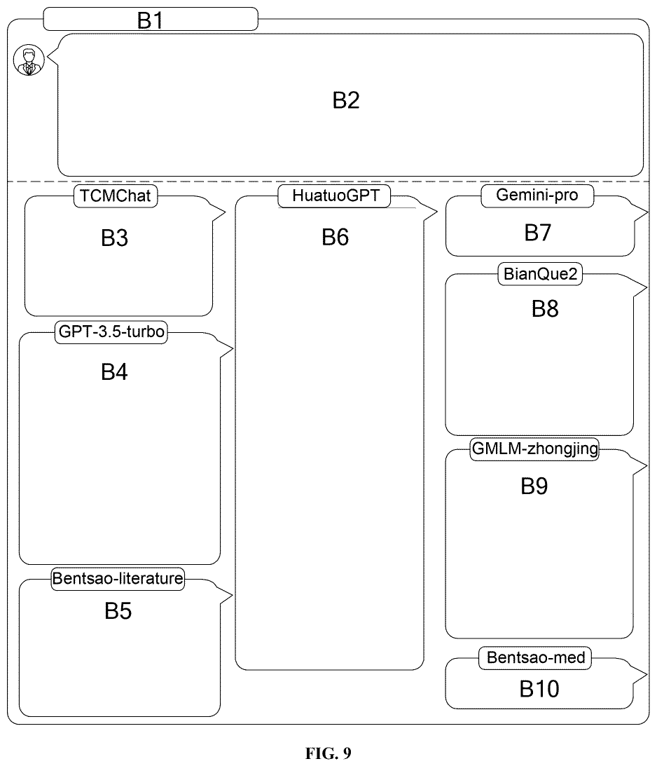

This example is a case on a task of extracting reading comprehension information of TCM. As shown in FIG. 9, response results of the Bentsao-med and the Bentsao-literature contain a significant dialogue error or duplicate text, which cannot meet a user need. The HuatuoGPT, the CMLM-Chongqing, and the BianQue2 do not find a cause of “mouth cracking and pain” from a context, but only explain a condition of the disease based on model cognition. The Gemini-pro can only partially see content in the context. Compared with other models, the GPT-3.5-turbo and the TCMChat can find an explanation related to the condition of the disease.

### Example 8

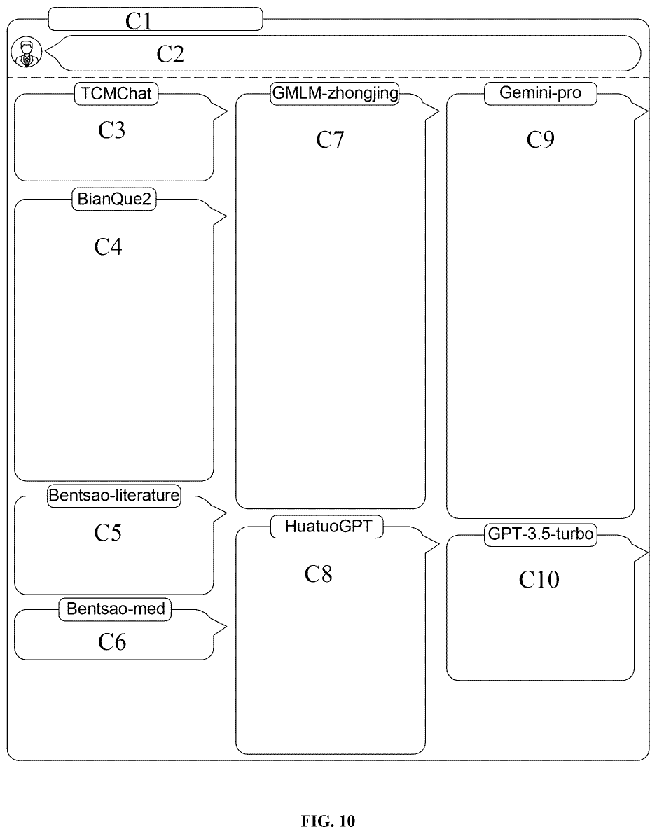

This example involves a case on intelligent recommendation of an “analogue compound”, as shown in FIG. 10. Responses of the Bentsao-med and the Bentsao-literature are not relevant to the question and do not provide any recommendation related to an analogical chemical composition spectrum. The HuatuoGPT attempts to provide some prescription suggestions, but these suggestions do not seem to be based on a consideration of the analogical chemical composition spectrum, lacking a factual basis and correlation. Suggestions from the BianQue2 and the CMLM-zhongjing also appear disorganized, without clear recommendation logic based on the analogical chemical composition spectrum. Although the Gemini-pro attempts to recommend a prescription based on efficacy or a pharmacological effect, this is unrelated to a topic of the question. A recommendation from the GPT-3.5-turbo also tends to be based on the efficacy or the pharmacological effect rather than directly on a chemical composition spectrum, which once again reflects that the model has a poor understanding of a concept of the “analogical chemical composition spectrum”. Compared with other models, the TCMChat performs the best. It can recommend a prescription that is on the ETCM website and whose chemical composition spectrum is similar to that of a given compound or prescription group. This demonstrates high accuracy and reliability of the TCMChat in understanding and handling such a question.

The trained Baichuan2-7B-Chat model in the present disclosure is compared with other models in existing technologies. If the trained Baichuan2-7B-Chat model outperforms any one of the other models in term of one or more of verification metrics (such as Accuracy, BLEU, METEOR, BertScore, MRR, and nDCG), the trained Baichuan2-7B-Chat model is considered the verified Baichuan2-7B-Chat model. Other models can be related models (such as the GPT-3.5-turbo, the Gemini-pro, the Bentsao-med, the Bentsao-literature, the BianQue2, the HuatuoGPT, and the CMLM-zhongjing), used for traditional Chinese medicine question answering in existing technologies.

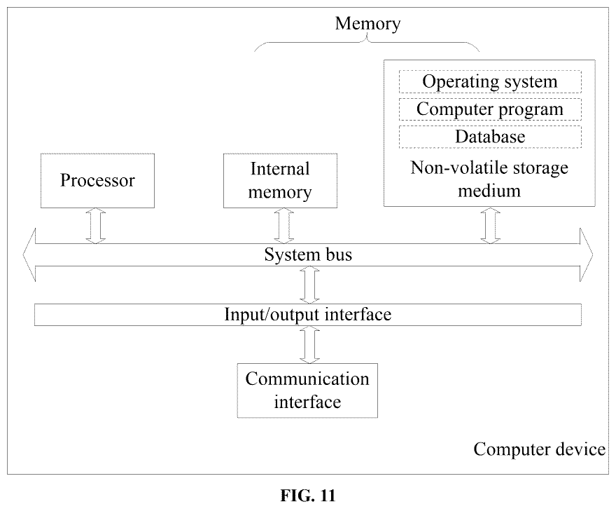

In an exemplary embodiment, a computer device is provided. The computer device may be a server or a terminal, and an internal structure thereof may be as shown in FIG. 11. The computer device includes a processor, a memory, an input/output (I/O) interface, and a communication interface. The processor, the memory, and the I/O interface are connected through a system bus. The communication interface is connected to the system bus through the I/O interface. The processor of the computer device is configured to provide computing and control capabilities. The memory of the computer device includes a non-volatile storage medium and an internal memory. The non-volatile storage medium stores an operating system, a computer program, and a database. The internal memory provides an environment for running the operating system and the computer program in the non-volatile storage medium. The database of the computer device is configured to store complementary definite data. The I/O interface of the computer device is configured to exchange information between the processor and an external device. The communication interface of the computer device is configured to connect to and communicate with an external terminal through a network. The computer program is executed by the processor to implement an LLM-based question answering method for TCM.

Those skilled in the art may understand that the structure shown in FIG. 11 is only a partial structure related to the solutions of the present disclosure and does not constitute a limitation on the computer device to which the solutions of the present disclosure are applied. Specifically, the computer device may include more or less components than those shown in the figure, or combine some components, or have different component arrangements.

In an exemplary embodiment, a computer device is further provided, including a memory and a processor. The memory stores a computer program, and the computer program is executed by the processor to implement the steps in the embodiments of the above LLM-based question answering method for TCM.

In an exemplary embodiment, a computer-readable storage medium is provided, which stores a computer program. The computer program is executed by a processor to implement the steps in the embodiments of the above LLM-based question answering method for TCM.

In an exemplary embodiment, a computer program product is provided, including a computer program. The computer program is executed by a processor to implement the steps in the embodiments of the above LLM-based question answering method for TCM.

Those of ordinary skill in the art may understand that all or some of the procedures in the method of the foregoing embodiments may be implemented by a computer program instructing related hardware. The computer program may be stored in a non-volatile computer-readable storage medium. When the computer program is executed, the procedures in the embodiments of the above method may be performed. Any reference to a memory, a database, or other media used in the embodiments of the present disclosure may include at least one of a non-volatile memory and a volatile memory. The non-volatile memory may include a read-only memory (ROM), a magnetic tape, a floppy disk, a flash memory, an optical memory, a high-density embedded non-volatile memory, a resistive random access memory (ReRAM), a magnetoresistive random access memory (MRAM), a ferroelectric random access memory (FRAM), a phase change memory (PCM), a graphene memory, and the like. The volatile memory may include a random access memory (RAM) or an external cache memory. As an illustration rather than a limitation, the RAM may be in various forms, such as a static random access memory (SRAM) or a dynamic random access memory (DRAM).

The database in the embodiments of the present disclosure may include at least one of a relational database and a non-relational database. The non-relational database may include a blockchain-based distributed database, but is not limited thereto. The processor in the embodiments of the present disclosure may be a general processor, a central processing unit (CPU), a graphics processor, a digital signal processor (DSP), and a programmable logic device, but is not limited thereto.

The technical characteristics of the above embodiments can be employed in arbitrary combinations. For brevity of description, all possible combinations of all the technical characteristics of the above embodiments may not be described; however, these combinations of the technical characteristics should be construed as falling within the scope defined by this specification as long as no contradiction occurs.

Several examples are used herein for illustration of the principles and implementations of the present disclosure. The description of the foregoing embodiments is used to help illustrate the method of the present disclosure and the core principles thereof. In addition, those of ordinary skill in the art can make various modifications in terms of specific implementations and a scope of application in accordance with the teachings of the present disclosure. In conclusion, the content of this specification shall not be construed as a limitation to the present disclosure.

What is claimed is:

1. A large language model (LLM)-based question answering method for traditional Chinese medicine (TCM), comprising:
building a TCM question answering device comprising one or more processors and a verified Baichuan2-7B-Chat model, comprising:
constructing a TCM knowledge database, wherein the TCM knowledge database comprises multi-source TCM knowledge data obtained from a book, literature, a network platform, and a TCM dataset;
generating unsupervised data and instruction data based on the TCM knowledge database, wherein application scenarios of the instruction data comprise a TCM knowledge base, a choice question, reading comprehension, entity extraction, medical case diagnosis, and TCM or prescription recommendation;
constructing a Baichuan2-7B-Chat model, wherein the Baichuan2-7B-Chat model is obtained by improving a transformer decoder, wherein an improvement method comprises: replacing a LayerNorm layer in the transformer decoder with an RMSNorm layer, replacing an absolute position embedding layer in the transformer decoder with a rotary position embedding layer, and replacing a ReLu activation function in the transformer decoder with a SwiGLU activation function;
performing unsupervised pre-training (PT) on the Baichuan2-7B-Chat model by using the unsupervised data to obtain a pre-trained Baichuan2-7B-Chat model;
performing supervised training on the pre-trained Baichuan2-7B-Chat model by using the unsupervised data and the instruction data to obtain a trained Baichuan2-7B-Chat model; and
setting a verification metric for each of the application scenarios, and verifying the trained Baichuan2-7B-Chat model to obtain the verified Baichuan2-7B-Chat model;
receiving, at the TCM question answering device from a client, information being one of the medical case diagnosis, the reading comprehension, and the TCM or prescription recommendation; and
outputting, at the TCM question answering device, a TCM answering result in a visualization manner by using the verified Baichuan2-7B-Chat model based on the information; wherein
for the choice question, the verification metric is accuracy of a selected answer;
for the reading comprehension, the verification metric comprises a bilingual evaluation understudy (BLEU) metric, a metric for evaluation of translation with explicit ordering (METEOR), an abstract evaluation metric, precision, a recall of an embedding of a generated text set, and a BertScore metric;
for the entity extraction, the verification metric comprises the precision, the recall of the embedding of the generated text set, and the BertScore metric;
for the medical case diagnosis, the verification metric comprises the accuracy of the selected answer, the precision, the recall of the embedding of the generated text set, and the BertScore metric;
for the TCM or prescription recommendation, the verification metric comprises a mean reciprocal rank (MRR), precision@K, and a normalized discounted cumulative gain (nDCG);
a formula for calculating the accuracy of the selected answer is as follows:
$$Accuracy = \frac{TP + TN}{TP + TN + FP + FN} ;$$
wherein Accuracy represents the accuracy of the selected answer, and TP, TN, FP, and FN respectively represent a number of true positive cases, a number of false positive cases, a number of false negative cases, and a number of true negative cases;
a formula for calculating the BLEU metric is as follows:
$$BLEU = BP \times exp ( {\sum }_{n = 1}^{N} {W}_{n} \times log {P}_{n} ) ;$$
wherein BLEU represents the BLEU metric; BP represents a penalty factor; N represents a size of a window for truncating continuous words; Wₙ represents a weight of an n^{th} phrase set within the window for truncating the continuous words; and Pₙ represents accuracy of the n^{th} phrase set, and the accuracy of the n^{th} phrase set is obtained by comparing various phrases in the n^{th} phrase set obtained by truncating a generated text and a reference text;
a formula for calculating the METEOR is as follows:
$$METEOR = ( 1 - Pen ) \times F ;$$
$$F = \frac{P \times R}{α \times P + ( 1 - α ) \times R} ;$$
$$Pen = γ * {( \frac{m}{ch} )}^{{β}_{1}} ;$$
wherein METEOR represents the METEOR, Pen represents a fragment penalty factor, F represents a harmonic mean of the accuracy and the recall, P represents accuracy of the generated text, R represents a recall of the generated text, α represents a parameter for adjusting weights of the P and the R, m represents a number of phrases allowed to be matched in the generated text, ch represents a number of phrases in the reference text, γ represents a proportional parameter, and β₁ represents a power parameter;
a formula for calculating the abstract evaluation metric is as follows:
$$Rouge - N = \frac{{\sum }_{S ∈ x} {\sum }_{{gram}_{n} ∈ S} {Count}_{match ( {gram}_{n} )}}{{\sum }_{S ∈ x} {\sum }_{{gram}_{n}} Count ( {gram}_{n} )} ;$$
$$ROUGE - L = \frac{( 1 + {β}_{2}^{2} ) \frac{LCS ( x , \hat{x} ) LCS ( x , \hat{x} )}{\begin{matrix}len ( x ) & len ( \hat{x} )\end{matrix}}}{\frac{LCS ( x , \hat{x} )}{len ( x )} + {β}_{2}^{2} \frac{LCS ( x , \hat{x} )}{len ( \hat{x} )}} ;$$
wherein Rouge−N represents a metric for performing abstract evaluation by taking the size N of the window for truncating the continuous words as a length, ROUGE−L represents a metric for performing the abstract evaluation based on a length of a longest common subsequence of the reference text and the generated text, S represents the reference text in a reference text set, x represents the reference text set, gramₙ represents the n^{th} phrase set within the window for truncating the continuous words, Count_{match(gram}_{ₙ}₎ represents a number of successfully matched phrases in the gramₙ, Count_{(gram}_{ₙ}₎ represents a total number of phrases contained in the gramₙ, \hat{x} represents the generated text set, LCS(x,\hat{x}) represents a length of a longest common subsequence of the x and the \hat{x}, len( ) represents a length calculation formula, and β₂ represents an adjustment parameter;
a formula for calculating the precision is as follows:
$$Precision = \frac{1}{\vert \hat{x} ′ \vert} {\sum }_{{\hat{x}}_{j} ∈ \hat{x} ′} {max}_{{x}_{i} ∈ x ′} ( {x}_{i}^{T} \hat{{x}_{J}} ) ;$$
wherein Precision represents the precision, x′ represents an embedding of the reference text set, \hat{x}′ represents the embedding of the generated text set, \hat{x}ⱼ represents an embedding of a j^{th} sentence in the embedding of the generated text set, xᵢ represents an embedding of an i^{th} sentence in the embedding of the reference text set, and a superscript T represents transposition;
a formula for calculating the recall of the embedding of the generated text set is as follows:
$$Recall = \frac{1}{\vert x ′ \vert} {\sum }_{{x}_{i} ∈ x ′} {max}_{{\hat{x}}_{j} ∈ \hat{x} ′} ( {x}_{i}^{T} ) ;$$
wherein Recall represents the recall of the embedding of the generated text set; and
a formula for calculating the BertScore metric is as follows:
$$BertScore ( F 1 - score ) = 2 * \frac{Precision * Recall}{Precision + Recall} ;$$
wherein BertScore(F1−score) represents the BertScore metric;
a formula for calculating the MRR is as follows:
$$MRR = \frac{1}{\vert Q \vert} {\sum }_{q = 1}^{\vert Q \vert} \frac{1}{{rank}_{q}} ;$$
wherein MRR represents the MRR, |Q| represents a number of suggestions, and rank_{q} represents a q^{th} suggestion;
a formula for calculating the precision@K is as follows:
$$Precision @ K = \frac{TP @ K}{TP @ K + FN @ K} ;$$
wherein Precision@K represents the precision@K, TP@K represents a number of true positive cases in first K recommendations, and FN@K represents a number of true negative cases in the first K recommendations; and
a formula for calculating the nDCG is:
$$nDCG = \frac{{\sum }_{{P}_{1} = 1}^{{P}^{′}} \frac{{2}^{{rel}_{{p}_{1}}} - 1}{{log}_{2} ( {p}_{1} + 1 )}}{{\sum }_{{p}_{2} = 1}^{\vert REL \vert} \frac{{2}^{{rel}_{{p}_{2}}} - 1}{{log}_{2} ( {p}_{2} + 1 )}} ;$$
wherein nDCG represents the nDCG, P′ represents a recommendation list of the generated text, relₚ_{₁} represents a score of a correlation between a p₁^{th} recommendation in the P′ and a question, |REL| represents a recommendation list of the reference text, and relₚ_{₂} represents a score of a correlation between a p₂^{th} recommendation in the |REL| and the question.

2. The LLM-based question answering method for TCM according to claim 1, wherein a loss function used in a process of performing the unsupervised PT on the Baichuan2-7B-Chat model by using the unsupervised data and in a process of performing the supervised training on the pre-trained Baichuan2-7B-Chat model by using the unsupervised data and the instruction data is as follows:
$${L}_{CLM}^{( A )} = - \frac{1}{\vert A \vert} {\sum }_{k = 1}^{\vert A \vert} log {P}_{θ} ( {A}_{k} \vert {A}_{< k} ) ;$$
$$wherein {L}_{CLM}^{( A )}$$
represents the loss function, A represents a sentence sequence in the unsupervised data, |A| represents a length of the sentence sequence, and P_{θ}(Aₖ|A_{<k}) represents an operation of predicting a probability of a k^{th} word Aₖ by using a word A_{<k} before a k^{th} word when a parameter of the Baichuan2-7B-Chat model is θ.

3. The LLM-based question answering method for TCM according to claim 1, wherein DeepSpeed-based distributed training is adopted in both a process of performing the unsupervised PT on the Baichuan2-7B-Chat model by using the unsupervised data and a process of performing the supervised training on the pre-trained Baichuan2-7B-Chat model by using the unsupervised data and the instruction data.

4. An LLM-based question apparatus for TCM, comprising a backend and a frontend, wherein
the frontend is configured to interact with a client; and
the backend is configured to communicated with the frontend, and comprises a verified Baichuan2-7B-Chat model, wherein the verified Baichuan2-7B-Chat model is built as follows:
constructing a TCM knowledge database, wherein the TCM knowledge database comprises multi-source TCM knowledge data obtained from a book, literature, a network platform, and a TCM dataset;
generating unsupervised data and instruction data based on the TCM knowledge database, wherein application scenarios of the instruction data comprise a TCM knowledge base, a choice question, reading comprehension, entity extraction, medical case diagnosis, and TCM or prescription recommendation;
constructing a Baichuan2-7B-Chat model, wherein the Baichuan2-7B-Chat model is obtained by improving a transformer decoder, wherein an improvement method comprises: replacing a LayerNorm layer in the transformer decoder with an RMSNorm layer, replacing an absolute position embedding layer in the transformer decoder with a rotary position embedding layer, and replacing a ReLu activation function in the transformer decoder with a SwiGLU activation function;
performing unsupervised pre-training (PT) on the Baichuan2-7B-Chat model by using the unsupervised data to obtain a pre-trained Baichuan2-7B-Chat model;
performing supervised training on the pre-trained Baichuan2-7B-Chat model by using the unsupervised data and the instruction data to obtain a trained Baichuan2-7B-Chat model; and
setting a verification metric for each of the application scenarios, and verifying the trained Baichuan2-7B-Chat model to obtain the verified Baichuan2-7B-Chat model;
wherein the frontend is further configured to: receive, from the client, information being one of the medical case diagnosis, the reading comprehension, and the TCM or prescription recommendation; based on the information, send a request for a TCM answering result corresponding to the information, to the backend; and display the TCM answering result in response to receiving a reply from the backend;
the backend is further configured to: in response to receiving the request from the frontend, processing the request by the verified Baichuan2-7B-Chat model and sending the reply comprising the TCM answering result to the frontend; wherein
for the choice question, the verification metric is accuracy of a selected answer;
for the reading comprehension, the verification metric comprises a bilingual evaluation understudy (BLEU) metric, a metric for evaluation of translation with explicit ordering (METEOR), an abstract evaluation metric, precision, a recall of an embedding of a generated text set, and a BertScore metric;
for the entity extraction, the verification metric comprises the precision, the recall of the embedding of the generated text set, and the BertScore metric;
for the medical case diagnosis, the verification metric comprises the accuracy of the selected answer, the precision, the recall of the embedding of the generated text set, and the BertScore metric;
for the TCM or prescription recommendation, the verification metric comprises a mean reciprocal rank (MRR), precision@K, and a normalized discounted cumulative gain (nDCG);
a formula for calculating the accuracy of the selected answer is as follows:
$$Accuracy = \frac{TP + TN}{TP + TN + FP + FN} ;$$
wherein Accuracy represents the accuracy of the selected answer, and TP, TN, FP, and FN respectively represent a number of true positive cases, a number of false positive cases, a number of false negative cases, and a number of true negative cases;
a formula for calculating the BLEU metric is as follows:
$$BLEU = BP \times exp ( {\sum }_{n = 1}^{N} {W}_{n} \times log {P}_{n} ) ;$$
wherein BLEU represents the BLEU metric; BP represents a penalty factor; N represents a size of a window for truncating continuous words; Wₙ represents a weight of an n^{th} phrase set within the window for truncating the continuous words; and Pₙ represents accuracy of the n^{th} phrase set, and the accuracy of the n^{th} phrase set is obtained by comparing various phrases in the n^{th} phrase set obtained by truncating a generated text and a reference text;
a formula for calculating the METEOR is as follows:
$$METEOR = ( 1 - Pen ) \times F ;$$
$$F = \frac{P \times R}{α \times P + ( 1 - α ) \times R} ;$$
$$Pen = γ * {( \frac{m}{ch} )}^{{β}_{1}} ;$$
wherein METEOR represents the METEOR, Pen represents a fragment penalty factor, F represents a harmonic mean of the accuracy and the recall, P represents accuracy of the generated text, R represents a recall of the generated text, α represents a parameter for adjusting weights of the P and the R, m represents a number of phrases allowed to be matched in the generated text, ch represents a number of phrases in the reference text, γ represents a proportional parameter, and β₁ represents a power parameter;
a formula for calculating the abstract evaluation metric is as follows:
$$Rouge - N = \frac{{\sum }_{S ∈ x} {\sum }_{{gram}_{n} ∈ S} {Count}_{match ( {gram}_{n} )}}{{\sum }_{S ∈ x} {\sum }_{{gram}_{n}} {Count}_{( {gram}_{n} )}} ;$$
$$ROUGE - L = \frac{( 1 + {β}_{2}^{2} ) \frac{LCS ( x , \hat{x} ) LCS ( x , \hat{x} )}{len ( x ) len ( \hat{x} )}}{\frac{LCS ( x , \hat{x} )}{len ( x )} + {β}_{2}^{2} \frac{LCS ( x , \hat{x} )}{len ( \hat{x} )}}$$
wherein Rouge−N represents a metric for performing abstract evaluation by taking the size N of the window for truncating the continuous words as a length, ROUGE−L represents a metric for performing the abstract evaluation based on a length of a longest common subsequence of the reference text and the generated text, S represents the reference text in a reference text set, x represents the reference text set, gramₙ represents the n^{th} phrase set within the window for truncating the continuous words, Count_{match(gram}_{ₙ}₎ represents a number of successfully matched phrases in the gramₙ, Count_{(gram}_{ₙ}₎ represents a total number of phrases contained in the gramₙ, x represents the generated text set, LCS(x,\hat{x}) represents a length of a longest common subsequence of the x and the \hat{x}, len( ) represents a length calculation formula, and β₂ represents an adjustment parameter;
a formula for calculating the precision is as follows:
$$Precision = \frac{1}{\vert {\hat{x}}^{′} \vert} {\sum }_{{\hat{x}}_{j} ∈ {\hat{x}}^{′}} {max}_{{x}_{i} ∈ {x}^{′}} ( {x}_{i}^{T} \hat{{x}_{j}} ) ;$$
wherein Precision represents the precision, x′ represents an embedding of the reference text set, \hat{x}′ represents the embedding of the generated text set, \hat{x}ⱼ represents an embedding of a j^{th} sentence in the embedding of the generated text set, xᵢ represents an embedding of an i^{th} sentence in the embedding of the reference text set, and a superscript T represents transposition;
a formula for calculating the recall of the embedding of the generated text set is as follows:
$$Recall = \frac{1}{\vert {x}^{′} \vert} {\sum }_{{x}_{i} ∈ {x}^{′}} {max}_{{\hat{x}}_{j} ∈ {\hat{x}}^{′}} ( {x}_{i}^{T} \hat{{x}_{j}} ) ;$$
wherein Recall represents the recall of the embedding of the generated text set; and
a formula for calculating the BertScore metric is as follows:
$$BertScore ( F 1 - score ) = 2 * \frac{Precision * Recall}{Precision + Recall} ;$$
wherein BertScore(F1−score) represents the BertScore metric;
a formula for calculating the MRR is as follows:
$$MRR = \frac{1}{\vert Q \vert} {\sum }_{q = 1}^{\vert Q \vert} \frac{1}{{rank}_{q}} ;$$
wherein MRR represents the MRR, |Q| represents a number of suggestions, and rank_{q} represents a q^{th} suggestion;
a formula for calculating the precision@K is as follows:
$$Precision @ K = \frac{TP @ K}{TP @ K + FN @ K} ;$$
wherein Precision@K represents the precision@K, TP@K represents a number of true positive cases in first K recommendations, and FN@K represents a number of true negative cases in the first K recommendations; and
a formula for calculating the nDCG is:
$$nDCG = \frac{{\sum }_{{p}_{1} = 1}^{{P}^{′}} \frac{{2}^{{rel}_{{p}_{1}}} - 1}{{log}_{2} ( {p}_{1} + 1 )}}{{\sum }_{{p}_{2} = 1}^{\vert REL \vert} \frac{{2}^{{rel}_{{p}_{2}}} - 1}{{log}_{2} ( {p}_{2} + 1 )}} ;$$
wherein nDCG represents the nDCG, P′ represents a recommendation list of the generated text, relₚ_{₁} represents a score of a correlation between a p₁^{th} recommendation in the P′ and a question, |REL| represents a recommendation list of the reference text, and relₚ_{₂} represents a score of a correlation between a p₂^{th} recommendation in the |REL| and the question.

5. The LLM-based question apparatus for TCM according to claim 4, wherein a loss function used in a process of performing the unsupervised PT on the Baichuan2-7B-Chat model by using the unsupervised data and in a process of performing the supervised training on the pre-trained Baichuan2-7B-Chat model by using the unsupervised data and the instruction data is as follows:
$${L}_{CLM}^{( A )} = - \frac{1}{\vert A \vert} {\sum }_{k = 1}^{\vert A \vert} log {P}_{θ} ( {A}_{k} \vert {A}_{< k} ) ;$$
$$wherein {L}_{CLM}^{( A )}$$
represents the loss function, A represents a sentence sequence in the unsupervised data, |A| represents a length of the sentence sequence, and Pe(Aₖ|A_{<k}) represents an operation of predicting a probability of a k^{th} word Aₖ by using a word A_{<k} before a k^{th} word when a parameter of the Baichuan2-7B-Chat model is θ.

6. The LLM-based question apparatus for TCM according to claim 4, wherein DeepSpeed-based distributed training is adopted in both a process of performing the unsupervised PT on the Baichuan2-7B-Chat model by using the unsupervised data and a process of performing the supervised training on the pre-trained Baichuan2-7B-Chat model by using the unsupervised data and the instruction data.

7. A computer device, comprising: a memory, a processor, and a computer program stored in the memory and executable on the processor, wherein the processor executes the computer program to implement the LLM-based question answering method for TCM according to claim 1.

8. The computer device according to claim 7, wherein in the LLM-based question answering method for TCM, a loss function used in a process of performing the unsupervised PT on the Baichuan2-7B-Chat model by using the unsupervised data and in a process of performing the supervised training on the pre-trained Baichuan2-7B-Chat model by using the unsupervised data and the instruction data is as follows:
$${L}_{CLM}^{( A )} = - \frac{1}{\vert A \vert} {\sum }_{k = 1}^{\vert A \vert} log {P}_{θ} ( {A}_{k} \vert {A}_{< k} ) ;$$
$$wherein {L}_{CLM}^{( A )}$$
represents the loss function, A represents a sentence sequence in the unsupervised data, |A| represents a length of the sentence sequence, and P_{θ}(Aₖ|A_{<k}) represents an operation of predicting a probability of a k^{th} word Aₖ by using a word A_{<k} before a k^{th} word when a parameter of the Baichuan2-7B-Chat model is θ.

9. The computer device according to claim 7, wherein in the LLM-based question answering method for TCM, DeepSpeed-based distributed training is adopted in both a process of performing the unsupervised PT on the Baichuan2-7B-Chat model by using the unsupervised data and a process of performing the supervised training on the pre-trained Baichuan2-7B-Chat model by using the unsupervised data and the instruction data.

10. A non-transitory computer-readable storage medium, storing a computer program thereon, wherein the computer program is executed by a processor to implement the LLM-based question answering method for TCM according to claim 1.

11. The non-transitory computer-readable storage medium according to claim 10, wherein in the LLM-based question answering method for TCM, a loss function used in a process of performing the unsupervised PT on the Baichuan2-7B-Chat model by using the unsupervised data and in a process of performing the supervised training on the pre-trained Baichuan2-7B-Chat model by using the unsupervised data and the instruction data is as follows:
$${L}_{CLM}^{( A )} = - \frac{1}{\vert A \vert} {\sum }_{k = 1}^{\vert A \vert} log {P}_{θ} ( {A}_{k} \vert {A}_{< k} ) ;$$
$$wherein {L}_{CLM}^{( A )}$$
represents the loss function, A represents a sentence sequence in the unsupervised data, |A| represents a length of the sentence sequence, and P_{θ}(Aₖ|A_{<k}) represents an operation of predicting a probability of a k^{th} word Aₖ by using a word A_{<k} before a k^{th} word when a parameter of the Baichuan2-7B-Chat model is θ.

12. The non-transitory computer-readable storage medium according to claim 10, wherein in the LLM-based question answering method for TCM, DeepSpeed-based distributed training is adopted in both a process of performing the unsupervised PT on the Baichuan2-7B-Chat model by using the unsupervised data and a process of performing the supervised training on the pre-trained Baichuan2-7B-Chat model by using the unsupervised data and the instruction data.
